(19) United States
(12) Patent Application Publication
(10) Pub. No.: US 2026/0053631 A1
(43) Pub. Date: Feb. 26, 2026

(54) DYNAMIC SYNDESMOSIS IMPLANT SYSTEMS AND METHODS

(71) Applicant: Paragon 28, Inc., Englewood, CO (US)

(72) Inventors: Kevin FARLEY, Denver, CO (US); Kyle James HARTSON, Denver, CO (US); John ROACH, Westminster, CO (US)

(73) Assignee: Paragon 28, Inc., Englewood, CO (US)

(21) Appl. No.: 19/287,231

(22) Filed: Jul. 31, 2025

Related U.S. Application Data
(63) Continuation of application No. PCT/US2024/014433, filed on Feb. 5, 2024.
(60) Provisional application No. 63/483,091, filed on Feb. 3, 2023.
(60) Provisional application No. 63/622,967, filed on Jan. 19, 2024.

(51) Int. Cl. A61F 2/42; A61F 2/08; A61F 2/46

(52) U.S. Cl. CPC A61F 2/4202; A61F 2/4606; A61F 2002/0852; A61F 2220/0008

(57) ABSTRACT

An implant system for dynamic stabilization of a syndesmosis joint,. The system includes a soft bone anchor, a dynamic bone anchor and a tether coupled to and extending between the soft bone anchor and the dynamic bone anchor. The first and second retention portions of the tether are knotlessly coupled to the dynamic bone anchor, and an intermediate portion of the tether extends between the first and second retention portions and passes through the soft bone anchor. A medial portion of the intermediate portion extends into the dynamic bone anchor and is resiliently coupled to the dynamic bone anchor. The system further includes an insertion and tensioning instrument, the soft bone anchor being removably retained on a free end of a first portion of the instrument, and the dynamic bone anchor being removably retained on at least one of the first portion and a second portion of the instrument.

### CROSS REFERENCE TO RELATED APPLICATION

This continuation application claims priority benefit from International Patent Application No. PCT/US2024/014433, filed on Feb. 5, 2024, and entitled “Dynamic Syndesmosis Implant Systems and Methods,” which claims priority benefit from U.S. Provisional Patent Application No. 63/483,091, filed on Feb. 3, 2023, and entitled “Dynamic Syndesmosis Implant Systems and Methods” and U.S. Provisional Ser. No. 63/622,967, filed on Jan. 19, 2024, and entitled “Dynamic Syndesmosis Implant Systems and Methods” the disclosures of each of these applications are hereby incorporated herein by reference in their entirety.

### TECHNICAL FIELD

The present disclosure relates generally to orthopedic surgery related to dynamic stabilization of bones and/or tissue. More specifically, but not exclusively, the present disclosure relates to implant devices, systems, and methods that achieve dynamic stabilization of a syndesmosis joint.

### BACKGROUND OF THE INVENTION

A syndesmosis joint is a fibrous joint where two bones are connected by strong ligaments or membrane. The distal tibiofibular syndesmosis, between the fibula and tibia, is formed by three major ligaments: the anterior inferior tibiofibular ligament (AITFL), the posterior inferior tibiofibular ligament (PITFL), and the interosseous tibiofibular ligament (ITFL). A fourth ligament, the inferior transverse tibiofibular ligament, is congruent with the PITFL, but sometimes considered a separate ligament. While technically the syndesmosis is the joint, most literature describes a syndesmosis injury as affecting the syndesmotic ligaments,

Syndesmotic injuries most typically occur to the distal tibiofibular syndesmosis, and result from trauma (such as, but not limited to, sports injuries). Syndesmotic injuries can occur as a purely ligamentous injury, or in combination with a bone fracture, where the syndesmotic ligaments become disrupted, separated, or injured.

The current standard of care for syndesmotic injuries involves either rigid fixation with a screw, or a suture/tether-based constraint (stabilization) across the entire width of the syndesmosis joint. Rigid screw-based fixation is simple to implant and stabilizes the joint, but fails to allow any motion at all, as would normally exist physiologically. This limits the patient's range of motion, and unpredictable screw failure locations can result in damage to existing bone and/or patient pain.

Currently commercially available suture/tethered-based syndesmosis stabilization/constraint implant systems allow for limited motion of the joint, but fail to mimic the intact ligament structures of the syndesmosis in terms of attachment location and distance between the bones of the joint (e.g., the tibia and fibula). These current stabilization/suture-based syndesmotic implants typically involve drilling through the joint bones in their entirety (e.g., drilling through the fibula and the tibia lateral to medial for distal tibiofibular syndesmosis), with a first component of the implant being positioned on a surface of a first bone (e.g., the lateral surface of the fibula), a second component positioned on a surface of a second bone (e.g., the medial surface of the tibia), and the suture or other elastic/resilient-type tether extending therebetween.

However, current tethered constraints result in a necessary decrease in structural strength due to the through holes formed in the surgical technique for the implant, and do not fully fill or enclose the holes which are only partially filled by the suture/tether extending therethrough. Further, these current tethered constraints utilize relatively long sutures/tethers as they span the entire width of the syndesmotic joint.

Thus, new and improved dynamic syndesmosis stabilization devices, systems, and methods are needed to overcome the above-noted drawbacks of the currently available solutions for addressing syndesmotic injuries.

### SUMMARY OF THE INVENTION

The present disclosure is directed toward syndesmotic implants, systems and methods for use in dynamic stabilization of a syndesmotic joint (i.e., constrained and controlled movement and/or forces in the joint). While the implants, systems and methods disclosed herein (and described in detail below) are particularly advantageous for use in, and dynamic stabilization of, distal tibiofibular syndesmosis, the implants and methods may be configured and utilized for use with any joint, and in particular any syndesmotic joint. The dynamic stabilization syndesmotic implants, systems and methods provide an advantageous relatively short suture/tether length which does not extend entirely through the syndesmotic joint bones. For example, with respect to dynamic stabilization of the distal tibiofibular syndesmosis, the dynamic stabilization syndesmotic implants, systems and methods are advantageously configured such that the implant does not extend to the medial portion of the tibia. The suture/tether of the dynamic stabilization syndesmotic implants, systems and methods thus advantageously better resists stretching from a strength/load perspective and time perspective.

The dynamic stabilization syndesmotic implants, systems and methods also minimize the amount of bone removal (e.g., bone drilling) of the syndesmotic joint bones for through holes to accommodate the suture/tether of the system. For example, with respect to dynamic stabilization of the distal tibiofibular syndesmosis, the dynamic stabilization syndesmotic implants, systems and methods advantageously limits the amount of bone removal from the tibia. The suture/tether of the dynamic stabilization syndesmotic implants, systems and methods thus advantageously minimize the number of bone openings and the total amount of bone removal/disturbance, which may not be completely filled/plugged by the suture/tether of the implants, systems and methods.

In one aspect, the present disclosure provides an implant system for dynamic stabilization of a syndesmosis joint, comprising: a soft bone anchor; a dynamic bone anchor; and a tether coupled to and extending between the soft bone anchor and the dynamic bone anchor. The first and second retention portions of the tether are knotlessly coupled to the dynamic bone anchor, and an intermediate portion of the tether extending between the first and second retention portions passes through the soft bone anchor. A medial portion of the intermediate portion extends into the dynamic bone anchor and is resiliently coupled to the dynamic bone anchor.

In some embodiments, the soft anchor is configured to deform into a dense enlarged shape upon tensioning of the intermediate portion of the tether. In some embodiments, the soft anchor is configured as a suture anchor. In some embodiments, the dynamic bone anchor comprises a head portion at one end and a body portion at an opposing end, and the head portion and the body portion are cannulated.

In some embodiments, the first and second retention portions pass through the cannulation of the head portion and the body portion, and out apertures of the head portion. In some such embodiments, the dynamic bone anchor further comprises a resilient bumper and a movable tether post within the cannulation of the body portion. In some such embodiments, the resilient bumper and the tether post are cannulated, and the first and second retention portions pass through the cannulation of the resilient bumper and the tether post. In some such embodiments, the resilient bumper is retained between an end of the body portion and the tether post. In some such embodiments, the medial portion of the intermediate portion extends about a portion of the tether post and through the cannulation of the resilient bumper. In some such embodiments, the medial portion of the intermediate portion of the tether forms a loop extending from the soft bone anchor.

In some embodiments, the first retention portion extends through cannulation of the head portion, out a first aperture of the head portion, over a portion of the outer top side, down into a second aperture of the head portion into the cannulation thereof, and back up through a fourth aperture of the head portion. In some such embodiments, the second retention portion extends through cannulation of the head portion, out the second aperture of the head portion, over a portion of the outer top side, down into the first aperture of the head portion into the cannulation thereof, and back up through a third aperture of the head portion. In some such embodiments, the portions of the first and second retention portions that extend over the portions of the outer top side form first and second retention loops, respectively, positioned past the outer top side of the head portion. In some such embodiments, the portions of the first and second retention portions that extend from the fourth and third apertures, respectively, pass under at least one of the first and second retention loops.

In some embodiments, the first and second apertures are open at respective lateral sides of the head portion. In some such embodiments, the third and fourth apertures are enclosed at the top side of the head portion by the top side of the head portion.

In some embodiments, an enlarged top portion of the head portion comprises an oblong cross-sectional shape. In some such embodiments, the enlarged top portion of the head portion comprises first and second lateral side portions that extend laterally past a base portion of the head portion and comprise a tapered underside, and third and fourth lateral side portions that extend between the first and second sides that do not extend laterally past the base portion.

In some embodiments, the tether is a suture.

In some such embodiments, the system further comprises an insertion and tensioning instrument, the soft bone anchor being removably retained on a free end of a first portion of the instrument, and the dynamic bone anchor being removably retained on at least one of the first portion and a second portion of the instrument.

In some embodiments, the first portion is removably coupled to the second portion. In some such embodiments, the first portion is removably coupled to the second portion via a laterally arranged dovetail coupling configuration.

In some embodiments, the first portion comprises a base portion that is removably coupled to a tip portion of the second portion, and a longitudinally elongated inserter portion extending longitudinally from the base portion to the free end. In some such embodiments, the free end of the inserter portion is forked. In some such embodiments, the forked free end of the inserter portion comprises a pair of tines and a base retention portion extending therebetween, and a pair of grooves extending longitudinally proximally from the base retention portion of the forked free end. In some such embodiments, end portions of the soft bone anchor extend over the grooves, and a medial portion of the soft bone anchor extends over the base retention portion between the pair of tines. In some such embodiments, the intermediate portion of the tether extends longitudinally from the soft bone anchor at the free end of the inserter portion along the inserter portion to the dynamic bone anchor.

In some embodiments, a head portion of the dynamic bone anchor is retained on a recess of a tip portion of the second portion. In some such embodiments, a base portion of the first portion extends partially over the recess on the tip portion of the second portion when the first portion is removably coupled to the second portion, and at least a portion of at least the head portion is positioned between the recess and the base portion when the first portion is removably coupled to the second portion to removably retain the dynamic bone anchor on the instrument.

In some embodiments, the second portion of the instrument comprises a main housing portion and a tension handle portion manually movably coupled with the main housing. In some such embodiments, the first and second retention portions of the tether are removably retained on the tension handle portion such that rotation of the tension handle portion relative to the main housing portion pulls the tether through the dynamic bone anchor and the soft bone anchor when the dynamic bone anchor is pulled against the instrument to shorten the length of and/or tension the intermediate portion extending between the dynamic bone anchor and the soft bone anchor. In some such embodiments, the first and second retention portions of the tether are removably retained on the tension handle portion such that rotation of the tension handle portion relative to the main housing portion pulls the tether through the main housing portion to pull the dynamic bone anchor against the instrument.

In some embodiments, the tension handle portion is longitudinally slidably coupled with the main housing portion, and longitudinal translation of the tension handle portion in a first direction extending away from a tip portion of the second portion of the instrument pulls the tether through the main housing portion to pull the dynamic bone anchor against the tip portion.

In some embodiments, the tension handle portion is longitudinally slidably coupled with the main housing portion, and longitudinal translation of the tension handle portion in a first longitudinal direction extending away from a tip portion of the second portion of the instrument pulls the tether through the dynamic bone anchor and the soft bone anchor when the dynamic bone anchor is pulled against the tip portion to shorten the length of and/or tension the intermediate portion extending between the dynamic bone anchor and the soft bone anchor. In some such embodiments, the second portion of the instrument further comprises at least one ratchet member coupled to the main housing portion comprising at least one tooth, the at least one ratchet member being resiliently biased against an externally threaded shaft portion of the tension handle portion such that the tension handle portion is rotatably coupled and longitudinally slidably coupled with the main housing portion. In some such embodiments, the at least one ratchet member prevents the tension handle portion from longitudinally slidably translating with respect to the main housing portion along a second longitudinal direction that opposes the first longitudinal direction.

In some such embodiments, the first and second retention portions of the tether comprise opposing end portions of the tether. In some such embodiments, the first and second retention portions of the tether each extend through a head portion of the dynamic bone anchor past an outer top side of the head portion.

In some embodiments, the first retention portion of the tether forms a first loop portion extending from a head portion of the dynamic bone anchor past an outer top side of the head portion, and a first end portion extending from the head portion past the outer top side, and the second retention portion of the tether forms a second loop portion extending from the head portion past the outer top side, and a second end portion extending from the head portion past the outer top side. In some such embodiments, the first end portion of the first retention portion of the tether extends over the outer top side and through the first loop portion and the second loop portion, and the second end portion of the first retention portion of the tether extends over the outer top side and through the first loop portion and the second loop portion to knotlessly couple the first and second retention portions to the dynamic bone anchor. In some such embodiments, the first retention portion of the tether extends through a first aperture of the head portion from an internal passageway of the head portion, over a portion of the outer top side, and back into the internal passageway via a second aperture of the head portion to form the first loop portion, and the first retention portion of the tether extends through the internal passageway from the second aperture, and through a third aperture of head portion past the outer top side to form the first end portion. In some such embodiments, the second retention portion of the tether extends through the second aperture of the head portion from the internal passageway, over a portion of the outer top side, and back into the internal passageway via the first aperture of the head portion to form the second loop portion, and the second retention portion of the tether extends through the internal passageway from the first aperture, and through a fourth aperture of head portion past the outer top side to form the second end portion.

In some embodiments, the first and second loop portions extend through a tip portion of the second portion and through a portion of the main housing portion to a carrier portion of the tension handle portion, and the first and second loop portions are releasably coupled with the carrier portion. In some such embodiments, movement of the tension handle portion relative to the main housing portion in a first direction extending away from the tip portion effectuates movement of the carrier portion in the first direction extending away from the tip portion to pull the first and second loop portions through the main housing portion and the head portion of the dynamic bone anchor against the tip portion. In some such embodiments, movement of the tension handle portion relative to the main housing portion in a first direction extending away from the tip portion effectuates movement of the carrier portion in the first direction extending away from the tip portion to enlarge the first and second loop portions and pull the tether through the dynamic bone anchor and the soft bone anchor when the dynamic bone anchor is pulled against the tip portion to shorten the length of and/or tension the intermediate portion extending between the dynamic bone anchor and the soft bone anchor.

In some embodiments, the carrier portion comprises a sleeve portion within the main housing portion that comprises at least one aperture, and post portion within the main housing portion that comprises at least one post that extends in a first direction extending away from the tip portion, and the sleeve portion and the post portion are biased into a first relative arrangement such that the at least one post extends through the at least one aperture and past an engagement side of a sleeve portion. In some such embodiments, the first and second loop portions extend around the at least one post and over the engagement side of the sleeve portion. In some such embodiments, the carrier portion further comprises a release member that is exposed at a back end of the tension handle portion and extends to the post portion, and the tension handle portion is configured such that translation of the release member in a second direction that opposes the first direction translates the post portion relative to the sleeve portion in the second direction such that the at least one post is positioned beneath the engagement side of the sleeve portion to release the first and second loop portions from the at least one post.

In some embodiments, the first and second retention portions are coupled to the tension handle portion, and the instrument is configured such that translation of the tension handle portion relative to the main housing portion tensions the first and second retention portions. In some such embodiments, the instrument is configurated such that the tensioning of the first and second retention portions via the tension handle portion pulls a head portion of the dynamic bone anchor against a tip portion of the second portion, and pulls the tether through the dynamic bone anchor and the soft bone anchor to shorten the length of and tension the intermediate portion extending between the dynamic bone anchor and the soft bone anchor. In some such embodiments, the tip portion of the second portion comprises a tip member longitudinally movably coupled with a front portion of the main housing portion, and the second portion further comprises a resilient member configured to resiliently resist movement of the tip portion relative to the main housing portion toward a rear portion of the main housing portion when the dynamic bone anchor is against the tip portion and the first and second retention portions are tensioned by the tension handle portion. In some such embodiments, the second portion further comprises a tension indicator longitudinally movably coupled with the main housing portion comprising an indicator portion with a visual indication that is visually exposed and adjacent to a reference portion of the main housing portion, and the tension indicator mates with the tip member such that the resiliently resisted movement of the tip portion relative to the main housing portion toward the rear portion thereof longitudinally translates the visual indication relative to the reference portion to provide a visual indication of the tension of the first and second retention portions, and thereby a visual indication of the tension of the intermediate portion extending between the dynamic bone anchor and the soft bone anchor.

In some embodiments, the second portion further comprises a tension indicator longitudinally translatably coupled with the main housing portion comprising an indicator portion with a visual indication that is visually exposed and adjacent to a reference portion of the main housing portion, and the tension of the first and second retention portions above a threshold longitudinally translates the visual indication relative to the reference portion to provide a visual indication of the tension of the first and second retention portions, and thereby a visual indication of the tension of the intermediate portion extending between the dynamic bone anchor and the soft bone anchor

In some such embodiments, first and second loop portions of the first and second retention portions, respectively, are coupled to the tension handle portion, and the instrument is configured such that translation of the tension handle portion relative to the main housing portion tensions the first and second retention portions. In some such embodiments, first and second end portions of the first and second retention portions, respectively, are fixed to the instrument. In some such embodiments, the first and second end portions are fixed to the instrument within the main housing portion, and are not fixed to the tension handle portion.

In some embodiments, a back end portion of the tension handle portion comprises a handle knob portion with a ring portion.

In one aspect, the present disclosure provides a method of dynamically stabilizing a distal tibiofibular syndesmosis joint. The method comprises obtaining any one of the implant systems described above, and implanting the soft bone anchor through a lateral fibula and into a cavity in a lateral cortex of a fibula such that the intermediate portion extends between the lateral fibula and the lateral cortex. The method also comprises tensioning the first and second retention portions to draw the dynamic bone anchor at least partially into the lateral cortex and to induce a tension in the intermediate portion extending between the dynamic bone anchor and the soft bone anchor to thereby induce tension across the distal tibiofibular syndesmosis joint.

In some such embodiments, implanting the soft bone anchor through the lateral fibula and into the cavity in the lateral cortex comprises implanting the soft bone anchor through the lateral fibula and into the cavity in the lateral cortex via manual manipulation of any one of the insertion and tensioning instruments as described above.

In some such embodiments, comprising removing the first portion from the second portion of the instrument, and decoupling the first portion and the soft bone anchor, after implanting the soft bone anchor through the lateral fibula and into the cavity in the lateral cortex,

In some such embodiments, tensioning the first and second retention portions comprises manually translating the tension handle portion and the main housing portion, of any one of the insertion and tensioning instruments as described above, relative to each other.

In some embodiments, tensioning the first and second retention portions translates a visual indicator of the instrument relative to a reference portion of the instrument to provide a visual indication of the tension of the first and second retention portions, and thereby a visual indication of the tension of the intermediate portion extending between the dynamic bone anchor and the soft bone anchor.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 illustrates a perspective view of an exemplary dynamic joint stabilization implant system, in accordance with an aspect of the present disclosure;

FIG. 2 illustrates another perspective side view of the implant system of FIG. 1, in accordance with an aspect of the present disclosure;

FIG. 3 illustrates a top view of the implant system of FIG. 1, in accordance with an aspect of the present disclosure;

FIG. 4 illustrates a side view of the implant system of FIG. 1, in accordance with an aspect of the present disclosure;

FIG. 5 illustrates another perspective view of the implant system of FIG. 1 with a pair of suture loops of the system in an extended arrangement, in accordance with an aspect of the present disclosure;

FIG. 6 illustrates a side cross-sectional exploded view of portions of the implant system of FIG. 1, in accordance with an aspect of the present disclosure;

FIG. 7 illustrates a perspective cross-sectional view of an anchor portion of the implant system of FIG. 1, in accordance with an aspect of the present disclosure;

FIG. 8 illustrates a perspective exploded view of the anchor portion of the implant system of FIG. 1, in accordance with an aspect of the present disclosure;

FIG. 9 illustrates a side exploded view of a portion of the anchor portion of the implant system of FIG. 1, in accordance with an aspect of the present disclosure;

FIG. 10 illustrates a side cross-sectional view of a portion of the anchor portion of the implant system of FIG. 1, in accordance with an aspect of the present disclosure;

FIG. 11 illustrates a perspective view of a head portion of the anchor portion of the implant system of FIG. 1, in accordance with an aspect of the present disclosure;

FIG. 12 illustrates another perspective view of the head portion of the anchor portion of the implant system of FIG. 1, in accordance with an aspect of the present disclosure;

FIG. 13 illustrates a side exploded view of the implant system of FIG. 1, in accordance with an aspect of the present disclosure;

FIG. 14 illustrates a side cross-sectional exploded view of the implant system of FIG. 1 as indicated in FIG. 13, in accordance with an aspect of the present disclosure;

FIG. 15 illustrates a cross-sectional view of the head portion of the anchor portion of the implant system of FIG. 1 as indicated in FIG. 13, in accordance with an aspect of the present disclosure;

FIG. 16 illustrates another cross-sectional view of the head portion of the anchor portion of the implant system of FIG. 1 as indicated in FIG. 13, in accordance with an aspect of the present disclosure;

FIG. 17 illustrates a side view of the implant system of FIG. 1 with the pair of suture loops of the system in an extended arrangement, in accordance with an aspect of the present disclosure;

FIG. 18 illustrates a cross-sectional view of the suture loop portion of the anchor portion of the implant system of FIG. 1 as indicated in FIG. 17, in accordance with an aspect of the present disclosure;

FIGS. 19-27 illustrates perspective views of an implant system insertion and tensioning instrument for implanting and utilizing the implant system of FIG. 1, in accordance with an aspect of the present disclosure;

FIG. 28 illustrates perspective view of a distal tibiofibular syndesmosis being dynamically stabilized via the implant, in accordance with an aspect of the present disclosure;

FIG. 29 illustrates a perspective view of another exemplary dynamic joint stabilization implant system with a pair of suture loops of a suture portion of the implant system in a reduced arrangement, in accordance with an aspect of the present disclosure;

FIG. 30 illustrates another perspective view of the implant system of FIG. 29, in accordance with an aspect of the present disclosure;

FIG. 31 illustrates a top view of the implant system of FIG. 29, in accordance with an aspect of the present disclosure;

FIG. 32 illustrates a side view of the implant system of FIG. 29, in accordance with an aspect of the present disclosure;

FIG. 33 illustrates an end view of the implant system of FIG. 1, in accordance with an aspect of the present disclosure;

FIG. 34 illustrates a perspective cross-sectional view of an anchor portion of the implant system of FIG. 29, in accordance with an aspect of the present disclosure;

FIG. 35 illustrates an end perspective view of the anchor portion of the implant system of FIG. 29 with a head portion thereof being partially transparent, in accordance with an aspect of the present disclosure;

FIG. 36 illustrates a side perspective view of the anchor portion of the implant system of FIG. 29 with the head portion thereof being partially transparent, in accordance with an aspect of the present disclosure;

FIG. 37 illustrates another end perspective view of the anchor portion of the implant system of FIG. 29, in accordance with an aspect of the present disclosure;

FIG. 38 illustrates an end view of the anchor portion of the implant system of FIG. 29 without the suture portion of the implant system, in accordance with an aspect of the present disclosure;

FIG. 39 illustrates another perspective view of the implant system of FIG. 29 with the pair of suture loops of the suture portion of the implant system in an extended pre-installed arrangement, in accordance with an aspect of the present disclosure;

FIG. 40 illustrates a perspective view of another exemplary implant system insertion and tensioning instrument for implanting and utilizing the implant system of FIGS. 29-39, in accordance with an aspect of the present disclosure;

FIG. 41 illustrates another perspective view of the insertion and tensioning instrument of FIG. 40, in accordance with an aspect of the present disclosure;

FIG. 42 illustrates an exploded perspective view of a portion the insertion and tensioning instrument of FIG. 40, in accordance with an aspect of the present disclosure;

FIG. 43 illustrates an exploded perspective view of another portion of the insertion and tensioning instrument of FIG. 40, in accordance with an aspect of the present disclosure;

FIG. 44 illustrates an exploded perspective view of another portion of the insertion and tensioning instrument of FIG. 40, in accordance with an aspect of the present disclosure;

FIG. 45 illustrates an exploded perspective view of another portion of the insertion and tensioning instrument of FIG. 40, in accordance with an aspect of the present disclosure;

FIG. 46 illustrates a perspective view of the implant system of FIGS. 29-39 loaded on the insertion and tensioning instrument of FIG. 40, in accordance with an aspect of the present disclosure;

FIG. 47 illustrates another perspective view of the implant system of FIGS. 29-39 loaded on the insertion and tensioning instrument of FIG. 40, in accordance with an aspect of the present disclosure;

FIG. 48 illustrates a perspective view of first and second portions of the insertion and tensioning instrument of FIG. 40, in accordance with an aspect of the present disclosure;

FIG. 49 illustrates another perspective view of first and second portions of the insertion and tensioning instrument of FIG. 40, in accordance with an aspect of the present disclosure;

FIG. 50 illustrates a perspective view of the first portion of the insertion and tensioning instrument of FIG. 40, in accordance with an aspect of the present disclosure;

FIG. 51 illustrates a perspective view of a forward portion of the second portion of the insertion and tensioning instrument of FIG. 40, in accordance with an aspect of the present disclosure;

FIG. 52 illustrates a perspective view of the implant system of FIGS. 29-39 loaded on the second portion of the insertion and tensioning instrument of FIG. 40 with a portion of the housing thereof removed, in accordance with an aspect of the present disclosure;

FIG. 53 illustrates a top view of the implant system of FIGS. 29-39 loaded on the second portion of the insertion and tensioning instrument of FIG. 40 with a portion of the housing thereof removed, in accordance with an aspect of the present disclosure;

FIG. 54 illustrates a top view of the implant system of FIGS. 29-39 loaded on the second portion of the insertion and tensioning instrument of FIG. 40 with a portion of the housing thereof removed and a tension handle portion thereof in a first actuated position, in accordance with an aspect of the present disclosure;

FIG. 55 illustrates a perspective view of the implant system of FIGS. 29-39 loaded on the second portion of the insertion and tensioning instrument of FIG. 40 with a portion of the housing thereof removed and the tension handle portion in the first actuated position, in accordance with an aspect of the present disclosure;

FIG. 56 illustrates a top view of the implant system of FIGS. 29-39 loaded on the second portion of the insertion and tensioning instrument of FIG. 40 with the tension handle portion in the first actuated position, in accordance with an aspect of the present disclosure;

FIG. 57 illustrates a top view of the implant system of FIGS. 29-39 loaded on the second portion of the insertion and tensioning instrument of FIG. 40 with the tension handle portion in a second actuated position that applies greater tension to the implant system than the first actuated position of the insertion and tensioning instrument and above a predefined threshold, in accordance with an aspect of the present disclosure;

FIG. 58 illustrates a top view of the implant system of FIGS. 29-39 loaded on the second portion of the insertion and tensioning instrument of FIG. 40 with a portion of the housing thereof removed and the tension handle portion in the second actuated position and applying the tension above the predefined threshold, in accordance with an aspect of the present disclosure;

FIG. 59 illustrates a perspective view of the implant system of FIGS. 29-39 loaded on the second portion of the insertion and tensioning instrument of FIG. 40 with a portion of the housing thereof removed and the tension handle portion in the second actuated position and applying the tension above the predefined threshold, in accordance with an aspect of the present disclosure;

FIG. 60 illustrates a perspective view of the implant system of FIGS. 29-39 being decoupled from the second portion of the insertion and tensioning instrument of FIG. 40 with a portion of the housing thereof removed, in accordance with an aspect of the present disclosure;

FIG. 61 illustrates a top cross-sectional view of a rear portion of the second portion of the insertion and tensioning instrument of FIG. 40 with a portion of the housing thereof removed, in accordance with an aspect of the present disclosure; and

FIG. 62 illustrates a top view of a rear portion of the second portion of the insertion and tensioning instrument of FIG. 40 with a portion of the housing thereof removed and a threaded shaft portion thereof as transparent, in accordance with an aspect of the present disclosure.

### DETAILED DESCRIPTION OF THE INVENTION

Generally stated, disclosed herein are devices and systems for achieving bone stabilization. Further, methods for using the devices and systems to achieve ligament fixation are discussed.

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current implants, devices, instrumentation and methods are described herein with reference to use with the bones of the ankle/leg, the bones of the foot, ankle and lower leg may be used to describe the surfaces, positions, directions or orientations of the implants, devices, instrumentation and methods. Further, the implants, devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the implants, devices, instrumentation and methods, and the aspects, components, features and the like thereof, described herein with respect to the right leg may be mirrored so that they likewise function with the left leg. Further, the implants, devices, instrumentation and methods, and the aspects, components, features and the like thereof, disclosed herein are described with respect to the leg for brevity purposes, but it should be understood that the implants, devices, instrumentation and methods may be used with other bones of the body having similar structures.

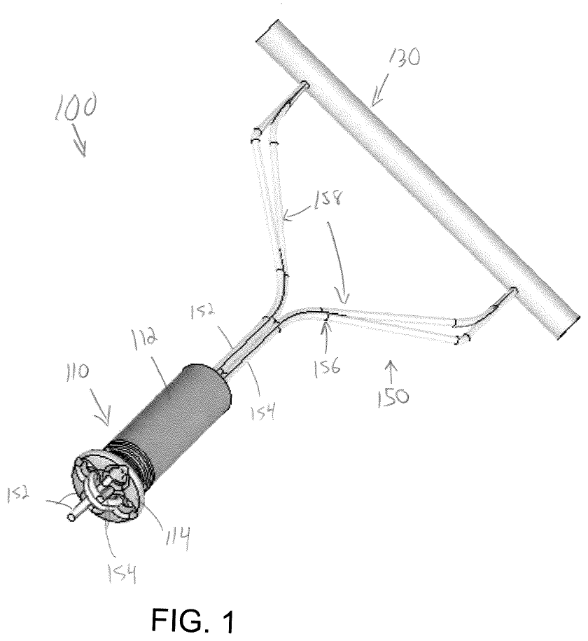

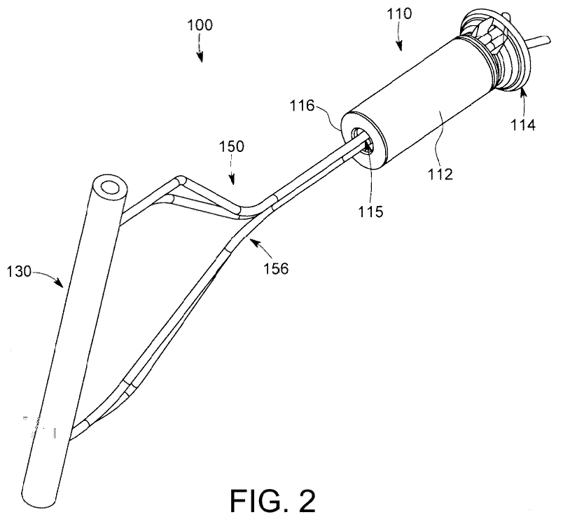

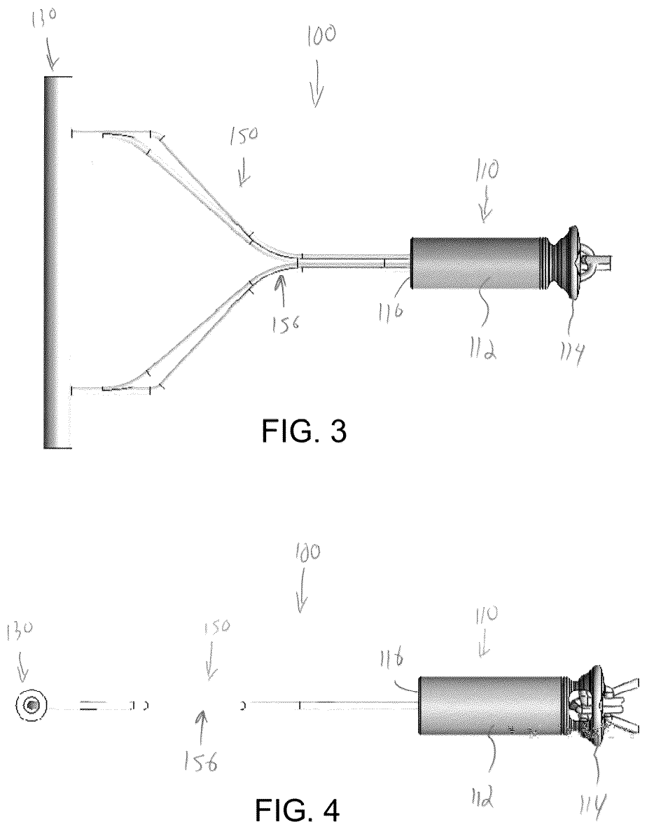

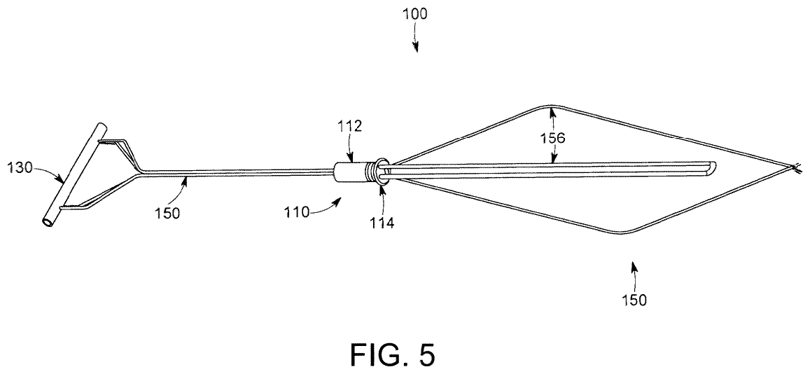

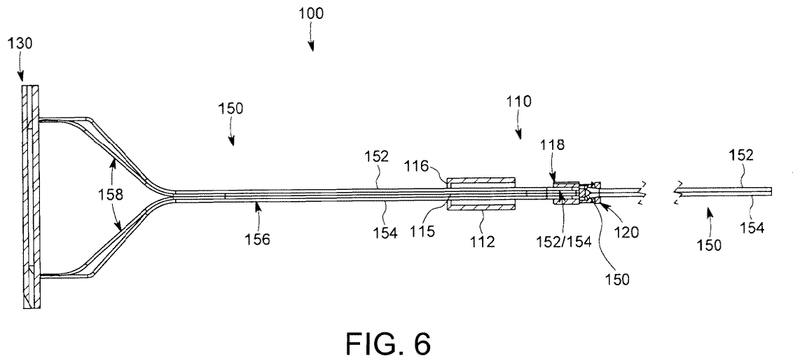

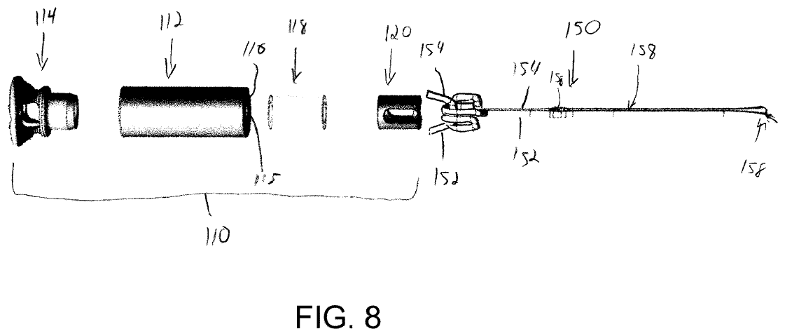

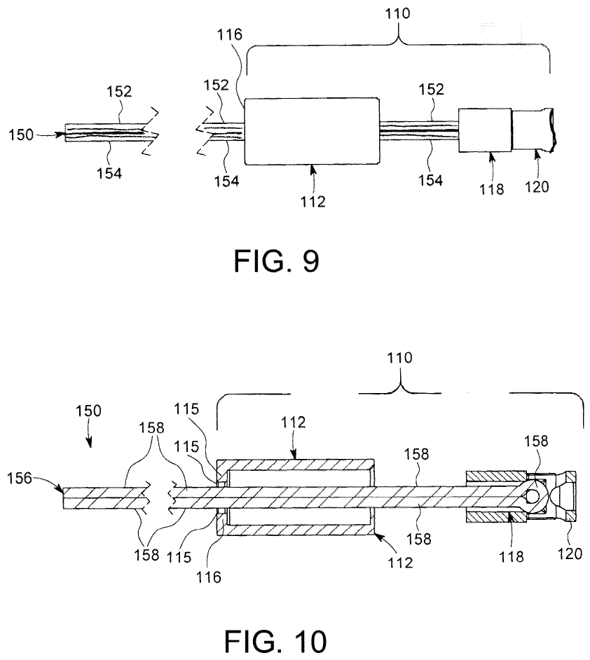

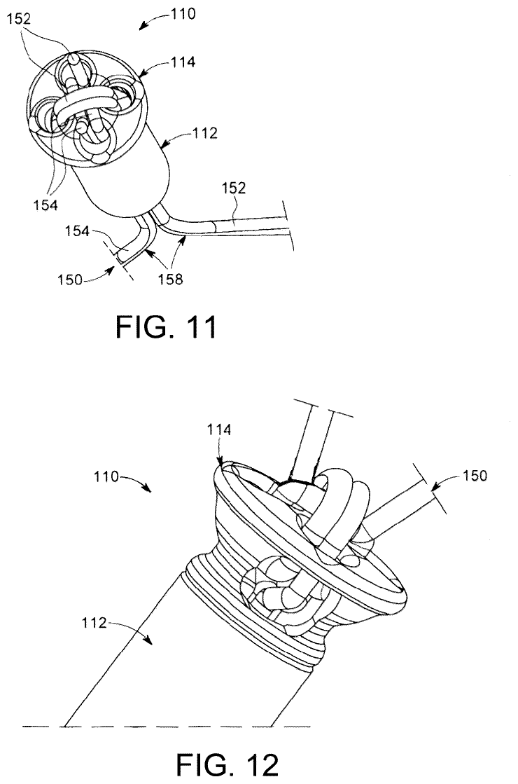

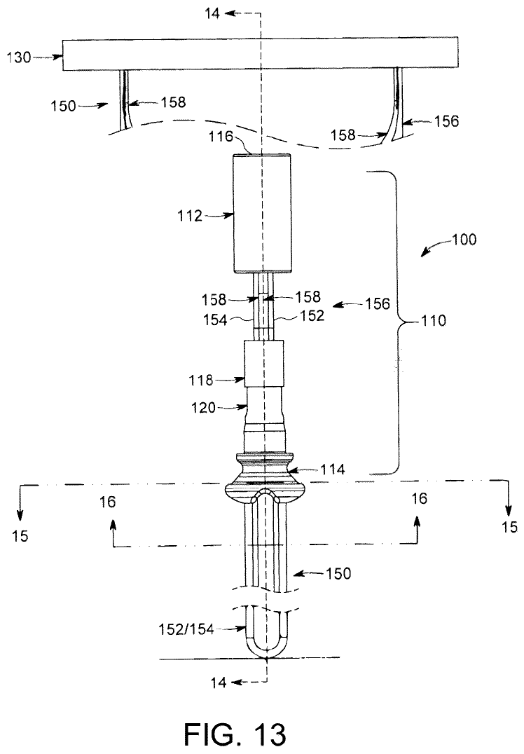

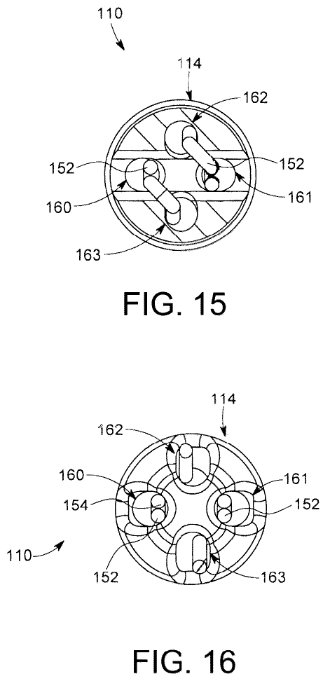

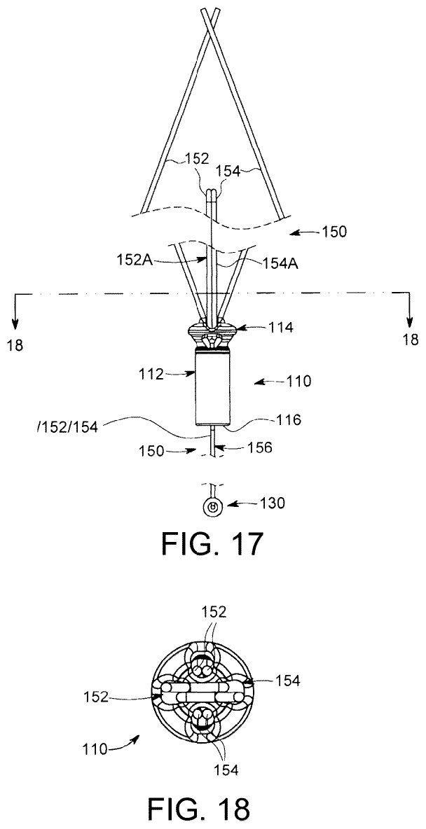

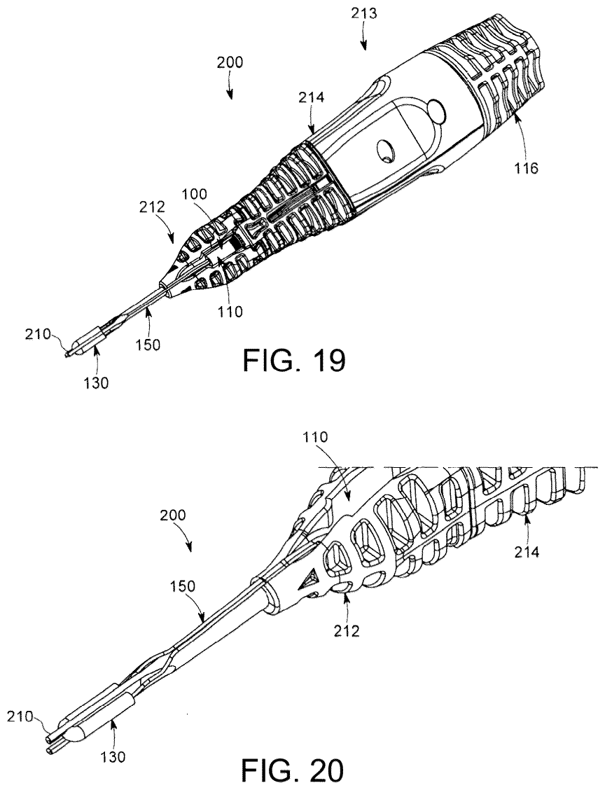

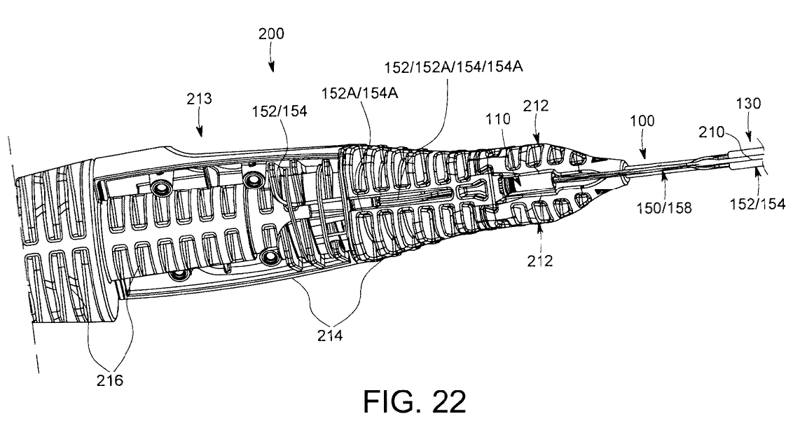

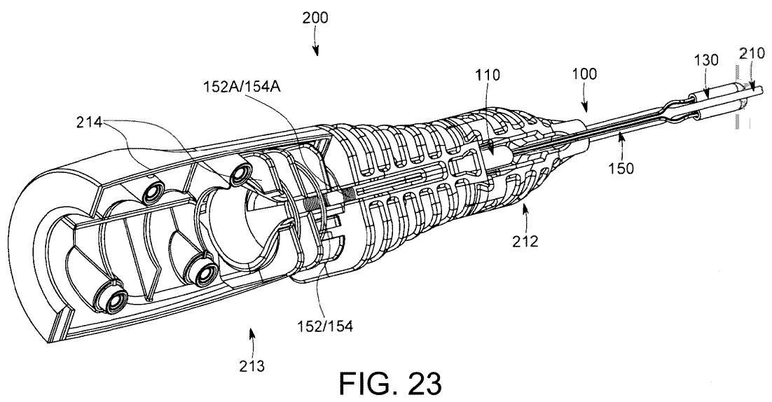

Referring to the drawings, wherein like reference numerals are used to indicate like or analogous components throughout the several views, and with particular reference to FIGS. 1-17 there is illustrated an implant system 100. The implant system 100 may be, for example, configured to heal syndesmotic ligaments post-operatively and/or stabilize syndesmotic joint, such as but not limited to a distal tibiofibular syndesmosis. The implant system 100 is configured to selectively constrain motion between two or more syndesmotic bones (e.g., in all directions) to allow for one or more syndesmotic ligaments extending therebetween to heal and/or to stabilize the syndesmotic joint. The implant system 100 allows for only a limited amount of relative movement/motion between bones in which it is implanted or installed, and such relative movement/motion and/or forces acting between the bones is be controlled (e.g., resisted/inhibited and/or encouraged/enhanced).

The components and portions of the implant system 100 may be made of, for example, titanium, stainless steel, polymers, polyester, polypropylene, UHMWPE, thermoplastic (e.g., thermoplastic urethane), bio-resorbable materials or any other biocompatible material so that the implant system 100 is configured to be implanted into a mammalian patient, such as a human patient.

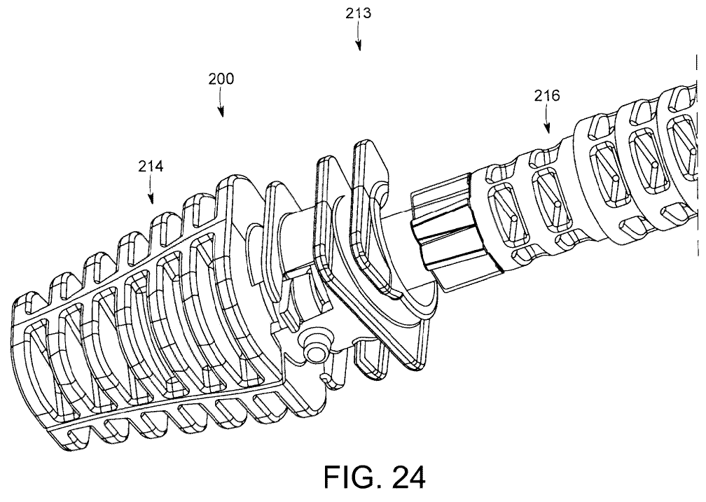

As shown in FIGS. 1-18, the implant system 100 provides for allow dynamic stabilization/fixation of a joint between two bones (either naturally distinct bones or bone portions/segments), such as bones of a syndesmotic joint (e.g., a distal fibula and tibia of a distal tibiofibular syndesmosis). The implant system 100 is configured to provide dynamic stabilization through constrained and/or controlled relative motion between the bones. The area of allowed constrained controlled motion provided by the implant system 100 may be set in a space or gap between adjacent bones or bone segments (such as the syndesmotic joint/space between the distal the fibula and tibia).

As shown in FIGS. 1-18, the implant system 100 includes a flexible suture or tether 150 as a constraint and/or tension member that extends through portions of, and from, a dynamic bone anchor 110. As also shown in FIGS. 1-18, a medial portion of the tether 150 extending from the dynamic bone anchor 110 passes through, or is otherwise coupled with, a bone anchor 130. The dynamic bone anchor 110 and the bone anchor 130 are configured to respectively be coupled to and implanted at least partially within first and second bones, such as bones of a syndesmotic joint (e.g., a distal fibula and tibia, respectively, of a distal tibiofibular syndesmosis), with the tether 150 extending between and coupled to the dynamic bone anchor 110 and the bone anchor 130.

The bone anchor 130 may be coupled with an intermediate loop portion 156 of the tether 150 extending from the dynamic bone anchor 110, and a medial loop portion 158 of the intermediate loop portion 156 and first and second retention portions 152, 154 of the tether 150 being knotlessly coupled to the dynamic bone anchor 110 which prevents the tether 150 from translating through the dynamic bone anchor 110 in a direction that enlarges the loop portion, as explained further below. As also explained below, the medial portion 158 of the intermediate loop portion 156 of the tether 150 is retained in the dynamic bone anchor 110 via an elastic or resilient member that provides dynamically controlled translation of the medial portion in a direction that enlarges the intermediate loop portion 156. The implant system 100 thereby provides dynamic stabilization of the joint between the first and second bones.

As shown in FIGS. 1-18, the intermediate loop portion 156 of the tether 150 may pass through the soft bone anchor 130 a plurality of times. For example, a first loop portion 162 of the intermediate loop portion 156 extending from the first retention portion 152 (within the bone anchor 130) may extend through a portion of the soft bone anchor 130 to the medial loop portion 158, and a second loop portion 164 of the intermediate loop portion 156 extending from the second retention portion 154 (within the bone anchor 130) may extend through a second portion of the soft bone anchor 130 to the medial loop portion 158. The first and second portions of the soft bone anchor 130 may be the substantially same portions of the soft bone anchor 130, or the portions may longitudinally/axially overlap or be spaced from each other.

In some embodiments, the first and second loop portions 162, 164 of the intermediate loop portion 156 of the tether 150 may extend through the soft bone anchor 130 to the medial loop portion 158 in differing longitudinal/axial directions. For example, as shown in FIGS. 1-18, the first and loop portion 162 may extend from the first retention portion 152 and into a first longitudinal/axial portion of the soft bone anchor 130, and through the soft bone anchor 130 to a second longitudinal/axial portion of the soft bone anchor 130, and the second and loop portion 164 may extend from the second retention portion 154 and into a third longitudinal/axial portion of the soft bone anchor 130 that is proximate to the second longitudinal/axial portion and distal to the first longitudinal/axial portion, and through the soft bone anchor 130 to a fourth longitudinal/axial portion of the soft bone anchor 130 that is proximate to the first longitudinal/axial portion and distal to the second longitudinal/axial portion. In this way, the first and second and loop portions 162, 164 may overlap as they extend longitudinally/axially within the soft bone anchor 130.

In some embodiments, the first and fourth portions of the soft bone anchor 130 are proximate to a first longitudinal/axial end of the soft anchor 130 and distal to a second longitudinal/axial end of the soft anchor 130, and the second and third portions of the soft bone anchor 130 are proximate to the second longitudinal/axial end and distal to a first longitudinal/axial end. In some embodiments, the first and fourth portions of the soft anchor 130 are the same portion of the soft anchor 130 or are positioned substantially adjacent to each other, and the second and third portions of the soft anchor 130 are the same portion of the soft anchor 130 or are positioned substantially adjacent to each other. As explained further below, the medial loop portion 158 extending between the first and second and loop portions 162 may extend into the dynamic bone anchor 110 and extend/be looped over/through a tether post 120 that is translatably retained within a cavity/lumen/cannulation of an anchor body 112 of the dynamic bone anchor 110. Movement of the tether post 120 within the anchor body 112 thereby either lengthens or shortens the size/length of the intermediate loop portion 156 to adjust the distance between the soft bone anchor 130 and the dynamic bone anchor 110. It is noted that the tether 150 may be slidably coupled with/through the soft bone anchor 130 to allow the soft bone anchor 130 to slide along the intermediate loop portion 156 of the tether 150 as it adjusts in size/length.

The tether 150 may be configured to mimic the function, location and/or length of an interosseous ligament, for example. The tether 150 may be of any thin, long and substantially freely manually flexible configuration or structure, such as a suture, strand, cable or string-like configuration. The tether 150 may be, for example, a biomedical suture or tether (e.g., a stranded cerclage cable), or similar construct. In some embodiments, the tether 150 may be formed of, for example, polymer, polyester, polypropylene or UHMWPE suture, strands or filaments, braids thereof, or a like material, as known by one of ordinary skill in the art. The tether 150 may be, for example, a suture (e.g., a braided suture), such as a single cross-section strand of suture or multiple loops of suture. For example, the tether 150 may be a UHMWPE and polypropylene co-braid suture.

The soft deformable anchor 130 may be solid or porous. For example, the soft deformable anchor 130 may be molded via a solid material. As another example, the soft deformable anchor 130 may be woven from one or more threads, fibers or filaments. In some embodiments, the soft deformable anchor 130 may be formed via one or more machining, additive and/or extrusion formation process. The soft deformable anchor 130 may be of one-piece construction or formed of a plurality of members or portions (and/or materials). The soft deformable anchor 130 may be formed or made from any biocompatible material(s). For example, the soft deformable anchor 130 may be made or formed of polyester, polyethylene (e.g., ultra-high-molecular-weight polyethylene), polypropylene, nylon (e.g., poylamide), silk, polyglycolic acid (PGA), polydioxanone (PDO), polylactic acid (PLA), polylactic-co-glycolic acid (PLGA), stainless steel, or a combination thereof. In one example, the soft deformable anchor 130 is formed of polyester.

The tether 150 may or may not be elastically axially/longitudinally stretchable or deformable. In some embodiments, the tether 150 may have one or more openings or gaps along its length that is configure to allow another tether or suture (or the like) to pass therethrough. In this way, the tether 150 my act as a tether or suture passer to translate another tether or suture to and/or through at least a portion of the implant system 100.

As explained above, the intermediate or medial loop portion 158 of the tether 150 extends through the bone anchor 130. As shown in FIGS. 1-18, in the illustrated embodiments, the bone anchor 130 is a soft deformable bone anchor, such as an all-soft suture anchor (ASAs). For example, the bone anchor 130 may be a tube, sleeve or tape made of suture or tether material (e.g., woven) through which the tether 150 is woven. In some other embodiments, the tube, sleeve or tape may be solid and/or formed of a resilient material, such as an elastomer.

The soft deformable anchor 130 may be elongated along a longitudinal direction in a neutral state. In some embodiments, the soft deformable anchor 130 may define a longitudinal length within the range of 5 mm and 35 mm. The soft deformable anchor 130 may be a size proportion bigger than the tether 150. For example, the width or diameter of the soft deformable anchor 130 may be larger than that of the tether 150.

In some embodiments, the soft deformable anchor 130 may be formed of an annular side wall that defines an inner cavity and first and second longitudinal ends. The soft deformable anchor 130 may thereby be hollow. The size of the inner cavity may be larger than the tether 150 such that the tether 150 is able to pass though the inner cavity. The inner cavity may extend through the soft deformable anchor 130 such that the inner cavity is open or accessible at one or both of the longitudinal ends, or the longitudinal ends may be closed or sealed. In some embodiments, the soft deformable anchor 130 may not define or include an inner cavity.

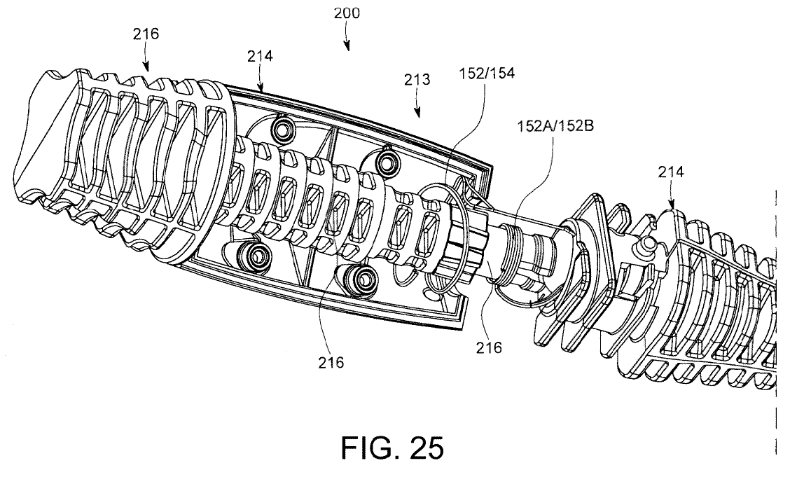

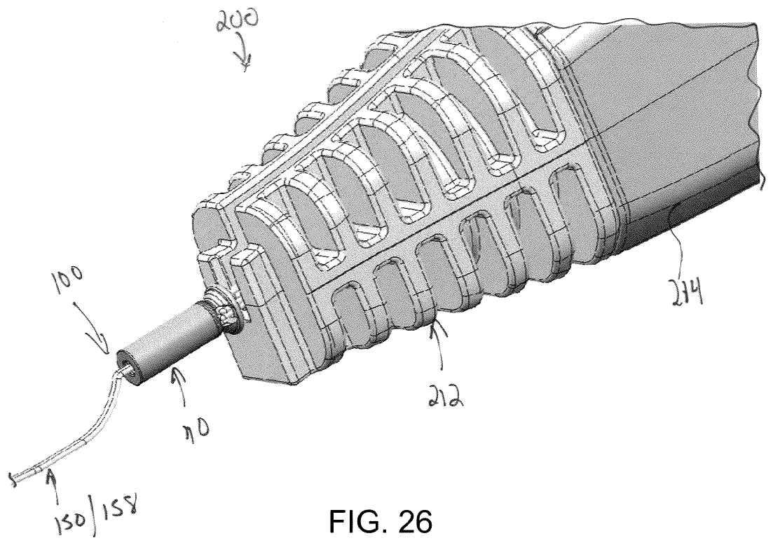

The soft deformable anchor 130 is flexible, collapsible, deformable, bendable, stretchable or otherwise re-arrangeable in overall shape. In a natural, neutral or non-deformed state, as shown in FIGS. 1-18, the soft deformable anchor 130 may extend substantially linearly or generally arcuately along the longitudinal direction. The soft deformable anchor 130 is configured such that tension on the portions on the suture extending from the soft deformable anchor 130 in a lateral direction that is angled from the longitudinal direction (e.g., substantially perpendicularly from the outer side surface of the anchor 130) deforms the soft deformable anchor 130 into a curved, bent-over, compressed or “bunched” shape, as shown in FIG. 28. The tether 150 may extend through a side or portion of the soft deformable anchor 130 one or more times which, when tension in the lateral direction (at least generally), deforms the anchor 130 into a dense concave/convex, arcuate, folded, “C” or “U” and/or “V” shape as shown in FIG. 28. For example, in an installed or neutral state, the anchor 130 may be bent and/or pulled together (e.g., via the tether 150) such that it assumes a sharply arcuate (e.g., defined by a relatively small radius) U-shape or V-shape, as shown in FIGS. 19, 20 and 23. As shown in FIG. 28, the tether 150 may be tensioned laterally such that the tether 150 deforms, compresses or pulls together a side or portion of the soft deformable anchor 130 such that the deformable anchor 130 or a portion thereof bunches up, balls up or otherwise becomes more densely arranged in the sharply arcuate shape such that it defines a larger width, lateral/cross-sectional dimension, or other size. In this way, the soft deformable bone anchor 130 is configured such that when it is inserted into a properly sized/shaped hole, tunnel or cavity in a bone and the suture portions extending from the anchor 130 are tensioned/pulled at least generally laterally, the anchor 130 is cinched up and enlarged in at least one dimension such that the anchor 130 compress against the side wall of the hole/cavity in the bone, creating a dense “ball” that is squeezed within the hole/tunnel/cavity and, thereby, anchored therein. As explained further below, in some exemplary methods, for dynamic stabilization of a distal tibiofibular syndesmosis, a hole/tunnel/cavity can be formed in the distal tibia (which may be formed via drilling the distal tibia). The anchor 130 may thereby be a tibial anchor. It is noted that the configuration of the anchor 130 allows it to be placed in a bone hole/tunnel/cavity with a substantially smaller diameter (e.g., 1-3 mm), thereby preserving bone and allowing for more bone preservation, for example.

As noted above and shown in FIGS. 1-18, the implant system 100 includes a dynamic bone anchor 110 through which the medial loop portion 158 of the intermediate loop portion 156 and the first and second retention portions 152, 154 of the flexible suture or tether 150 extends. As also shown in FIGS. 1-18, a medial portion of the tether 150 extending from the dynamic bone anchor 110 passes through, or is otherwise coupled with, a bone anchor 130. As also noted above, the dynamic bone anchor 110 is configured to be coupled to and implanted at least partially within a second bone, such as a bone of a syndesmotic joint (e.g., a distal fibula of a distal tibiofibular syndesmosis), with the tether 150 extending between and coupled to the dynamic bone anchor 110 and the bone anchor 130.

With reference to in FIGS. 1-18, in some embodiments, the dynamic bone anchor 110 includes an anchor body 112 and a head portion 114. The anchor body 112 may be positioned at and/or define a first longitudinal/axial end or end portion 111 of the bone anchor 110, and the head portion 114 may be positioned at and/or define a second longitudinal/axial end or end portion 113 of the bone anchor 110. In some embodiments, the dynamic bone anchor 110 as a whole, may be substantially or generally cylindrical.

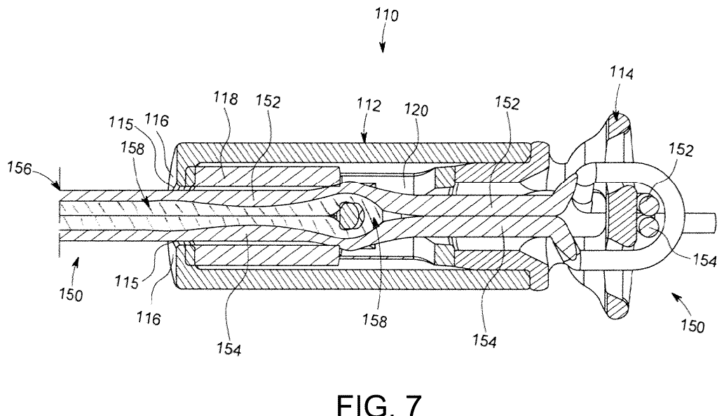

As shown in FIGS. 6, 7, 10 and 14, the anchor body 112 may include an inner cavity/lumen/cannulation/opening that extends longitudinally/axially therethrough. The inner cavity of the anchor body 112 is open at the first end 111 of the bone anchor 110 via a tether aperture 115 in an end wall 116 of the anchor body 112. The tether aperture 115 is configured to allow the medial loop portion 158 of the intermediate loop portion 156 and the first and second retention portions 152, 154 of the tether 150 to extend therethrough, as shown in FIGS. 6, 7, 10 and 14.

As shown in FIGS. 6-10, 13 and 14, the anchor body 112 contains a resilient/elastically deformable bumper 118 and a tether post 120 movably contained within the inner cavity thereof as the bumper 118 is larger than the tether aperture 115. The tether post 120 is at least axially/longitudinally translatable within the inner cavity of the anchor body 112. The bumper 118 is at least axially/longitudinally compressible and elongatable within the inner cavity of the anchor body 112. The bumper 118 may thereby be considered at least axially/longitudinally translatable within the inner cavity as at least some portions of the bumper 118 translate axially/longitudinally within the inner cavity as it axially/longitudinally compresses/shortens and elongates. The bumper 118 and/or the tether post 120 may also be rotatably retained within the inner cavity of the anchor body 112.

The bumper 118 is axially/longitudinally positioned and trapped between the end wall 116 of the anchor body 112 and the tether post 120, and thus is axially/longitudinally compressible and elongatable therebetween. The tether post 120 may abut an axial/longitudinal end of the bumper 118, and the other axial/longitudinal end of the bumper 118 may abut the end wall 116. As shown in FIGS. 6-10, 13 and 14, the bumper 118 may have a cavity/lumen/cannulation/opening that extends longitudinally/axially therethrough. The cannulation of the bumper 118 is configured to allow the intermediate loop portion 156 and the first and second retention portions 152, 154 of the tether 150 to extend axially/longitudinally therethrough and to and past the tether post 120, respectively.

In some embodiments, the bumper 118 may be an elastic or resilient spring-like member. The bumper 118 may be comprised of one or more elastically deformable member or material. For example, the bumper 118 may be one or more springs (e.g., disc or coil spring) or elastically compressible disc or tube, or a combination thereof. For example, the bumper 118 may comprise an elastically compressible disc (e.g., elastomeric, polymer, polyurethane or polyethylene disc), tube (e.g., a polyurethane tube) or coil spring. In some embodiments, the bumper 118 comprises at least one urethane tube or like member, such as at least one polycarbonate urethane (PCU) tube or like member or at least one thermoplastic polyurethane (TPU) tube or like member.

The bumper 118 is configured such that it elastically deforms to tension the medial loop portion 158 of the tether 150 that pulls the anchor portion 130 and the dynamic anchor 110 together, and/or allows a limited degree of relative movement between the anchor portion 130 and the dynamic anchor 110. As explained further below, the tether post 120 may be forced, variably, axially/longitudinally toward the end wall 116 of the anchor body 112 via tension on the medial loop portion 158 of the intermediate loop portion 156 of the tether 150 such that it is variably compresses the bumper 118 between the tether post 120 and the end wall 116 of the anchor body 112.

As shown in FIGS. 6-10, 13 and 14, the tether post 120 may have a cavity/lumen/cannulation/opening that extends longitudinally/axially therethrough. The cannulation of the tether post 120 is configured to allow the first and second retention portions 152, 154 of the tether 150 to extend axially/longitudinally therethrough, through/past the bumper 118, and to the intermediate loop portion 156.

As shown in FIGS. 1-18, the head portion 114 may be configured to mate with the anchor body 112 (or they may be one piece) and includes a cavity/lumen/cannulation/opening that extends longitudinally/axially therethrough that allows the first and second retention portions 152, 154 of the tether 150 to extend therethrough. The cannulation of the head portion 114 may be configured to allow the first and second retention portions 152, 154 to be retained, coupled, affixed or the like to the head portion 114 and each other such that the first and second retention portions 152, 154 are prevented from sliding or otherwise translating back through the dynamic anchor 110 to elongate/enlarge the intermediate loop portion 156 of the tether 150, thereby the medial loop portion 158 and the distance between the dynamic anchor 110 and the soft anchor 130.

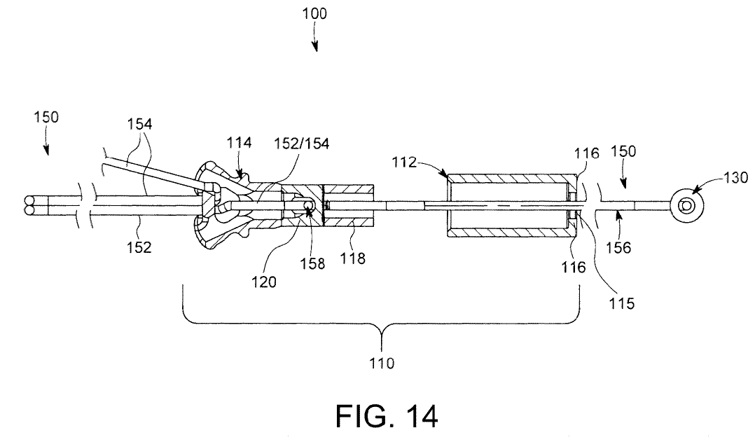

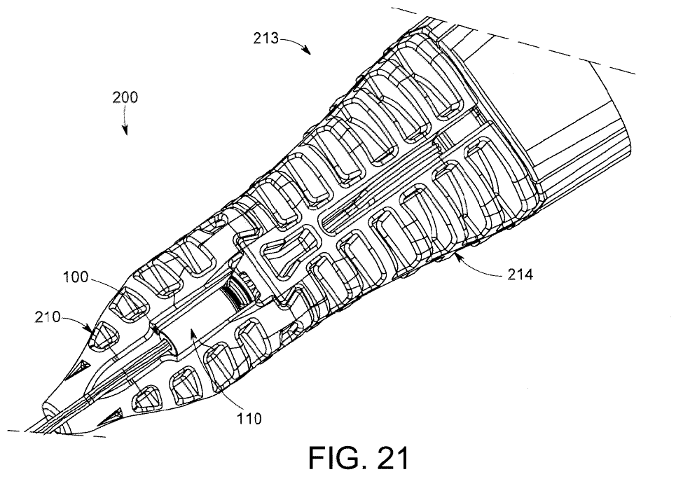

As shown in FIGS. 11-16, the head portion 114 may include a plurality of apertures that are in communication with the cannulation configured such that the first and second retention portions 152, 154 can extend through and around portions of the head portion 114. In some such embodiments, the first and second retention portions 152, 154 extend through and around portions of the head portion 114 in a knotless arrangement that secures or fixes the first and second retention portions 152, 154. For example, as shown in FIGS. 15-18, the head portion 114 may include a first aperture 160, a second aperture 161, a third aperture 162 and a fourth aperture 163. In the illustrated exemplary embodiment, the first retention portion 152 of the tether 150 extends through the first aperture 160 from the cannulation of the head portion 114, over the top side of the head portion 114 and down into the second aperture 161, and then back up through the fourth aperture 163, as shown in FIGS. 15-18. Similarly, the second retention portion 154 of the tether 150 extends through the second aperture 161 from the cannulation of the head portion 114, over the top side of the head portion 114 and down into the first aperture 160, and then back up through the third aperture 162, as shown in FIGS. 15-18. The pathway of the first and second retention portions 152, 154 thereby each form an adjustment loop 152A, 154A above the top side of the head portion 114. As also shown in FIGS. 15-18, the end portions of the first and second retention portions 152, 154 may pass under one or both of the adjustment loops 152A, 154A. In this way, when the tether 150 is tensioned, the adjustment loops 152A, 154A are pulled down onto/towards the top side of the head portion 114 and compress against end portions of the first and second retention portions 152, 154 to fix the tether 150 without the use of a knot.

The back or underside of the head portion 114 of the dynamic anchor 110 may be configured to mate with bone and/or a bone plate. For example, as shown in FIGS, 1-18, the head portion 114 a backside taper or that is configured to engage bone and/or a countersunk aperture of a bone plate, for example. In some other embodiments, the back or underside of the head portion 114 may include threads configured to engage bone and/or a threaded aperture of a bone plate, for example. The outer surface of the anchor body 112 may likewise be configured substantially smooth or with threads, for example.

As shown in FIGS. 19-27, the stabilization system 100 may be coupled to an insertion or implant instrument 200 that is configured to implant and use the stabilization system 100. As shown in FIGS. 19-23, the dynamic anchor 210 may be removably housed within a cavity in a first insertion portion 212 of the instrument 200, and the soft anchor 130 is removably coupled on/over a forked free end 210 of the first insertion portion 212. The soft anchor 130 may be removably retained on the forked free end 210 via tension of the tether 150, which may be removably coupled to a second portion 213 of the instrument 200, as discussed further below. As shown in FIGS. 19-23, the forked free end 210 of the first insertion portion 212 has a pair of tines and a base portion extending therebetween, and a pair of grooves extending proximally from the base portion of the forked free end. The soft anchor 130 is retained on the forked free end 210 such that end portions of the soft anchor 130 extend over or within the pair of grooves, and a medial portion of the soft anchor 130 extends over the base portion between the pair of tines.

The instrument may be utilized to introduce the soft anchor 130 into a cavity or hole in a first bone (e.g., lateral cortex of tibia) through a second bone (fibula). After insertion, the instrument may be pulled pack to tension the intermediate loop portion 156 and deform the soft anchor 130 to fix the soft anchor 130 on the bone. After such fixation, the first portion 212 can be removed or decoupled from the second portion 213 to expose the dynamic anchor 110, as shown in FIGS. 26 and 27.

As shown in FIGS. 22-25, the second portion 213 may include a proximal tensioning handle 216 and a main housing 214. The tensioning handle 216 may be removably rotatable coupled within a cavity of the main housing 214, and the adjustment loops 152A, 154A may be wrapped around or otherwise releasably coupled thereto, as shown in FIG. 25. The tensioning handle 216 and the main housing 214 may be configured such that the tensioning handle 216 can only be rotated one direction, such that rotation of the tensioning handle 216 pulls the adjustment loops 152A, 154A to apply shorten the intermediate loop portion 156 such that the dynamic anchor 110 is brought into contact with the second bone. The tensioning handle 216 may be further rotated to ultimately apply a desired level of tension across the joint of the first and second bones.

After the tension is applied, the second portion 213 may be configured such that the tensioning handle 216 is removed or decoupled from the main housing 214, and thereby release the adjustment loops 152A, 154A. It is noted that the static friction of the portions of the tether 150 on itself and the components of the system 100 are high enough that the tension is maintained and the adjustment loops 152A, 154A do not slide back into the head portion 114. As shown in FIGS. 22 and 23, the end portions of the first and second retention portions 152, 154 are couped, and housed and maintained, within/on the main housing 213. Accordingly, after the tension is applied and the tensioning handle 216 is removed, the main housing 213 can be pulled back to tension the end portions of the first and second retention portions 152, 154 to reduce the adjustment loops 152A, 154A onto the top side of the head portion 114 and secure the tether 150 (without a knot), as explained above. The first and second retention portions 152, 154 may then be cut close to the head portion 114 to remove the system fully from the instrument 200.

As shown in FIG. 28, the system 100 may be used with a bone plate 190 and bone screws 192 to stabilize a distal tibiofibular syndesmosis. As shown in FIG. 28, the bone plate may be coupled to a fibula 180, which screws 192 extending through apertures 191. One or more of the dynamic anchors 110 may be inserted through an aperture 191 and into the fibula 180. In some embodiments, the soft anchor 130 may be implanted in the tibia 182, and the tether 150 spanning the joint space to dynamically stabilize the joint. In some embodiments, the system 100 may be implemented in conjunction with a bone plate the same as or similar to the bone plate 190 and one or more other implants having dynamic properties and configured to be implanted at least partially in at least one of the tibia and/or fibula.

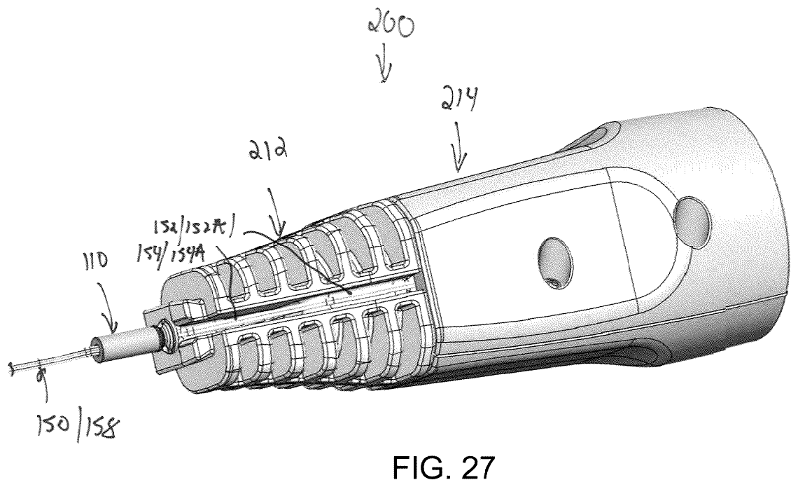

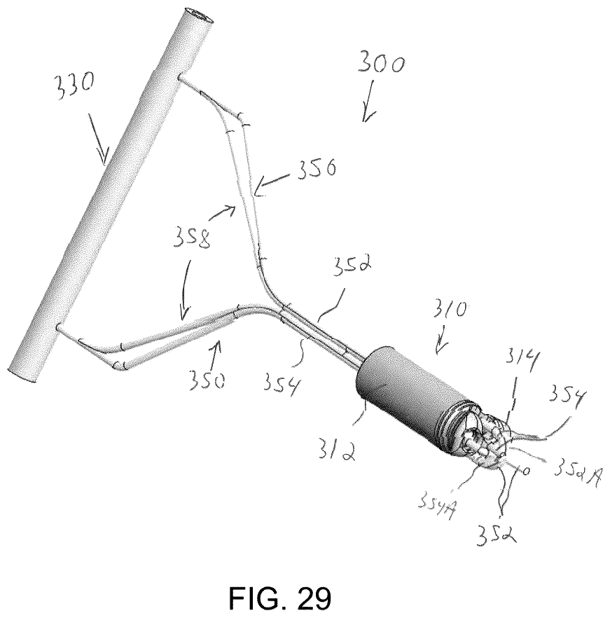

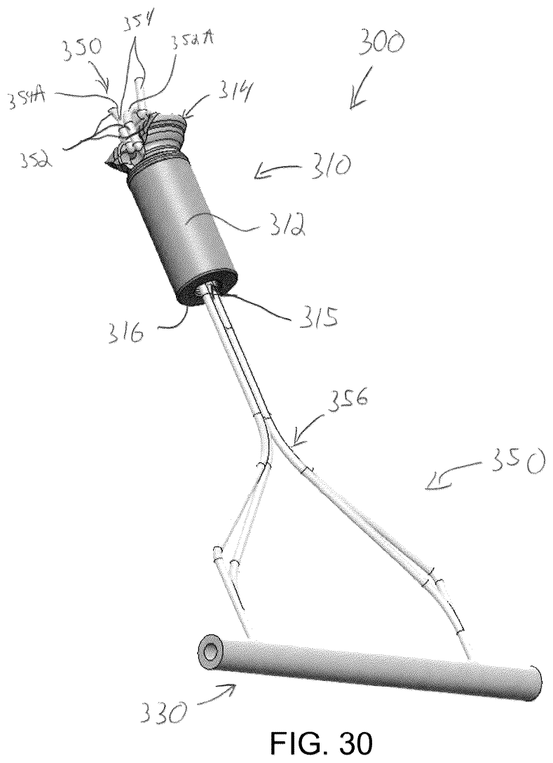

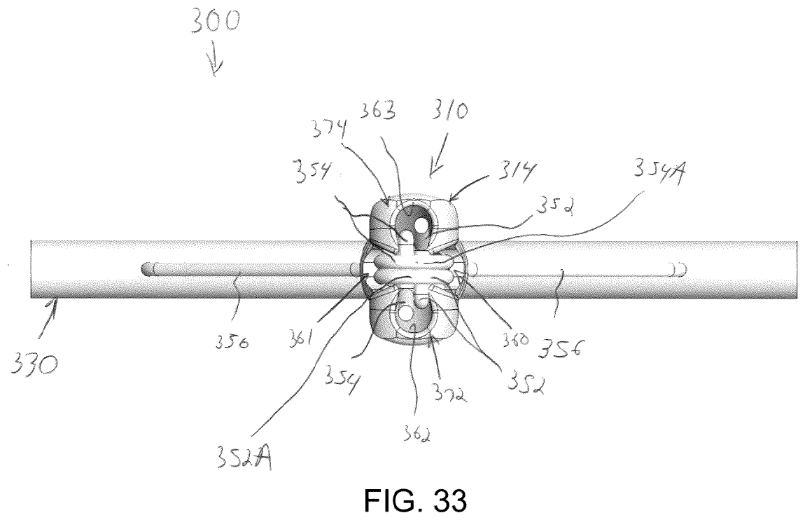

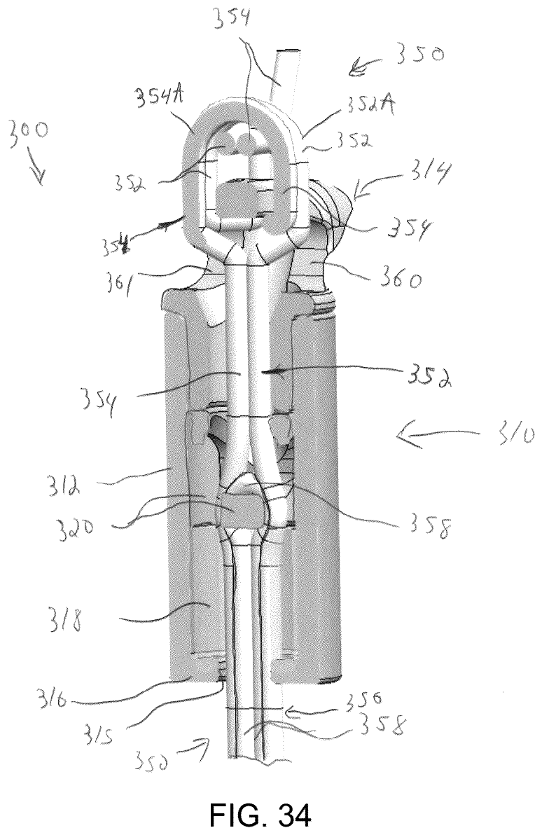

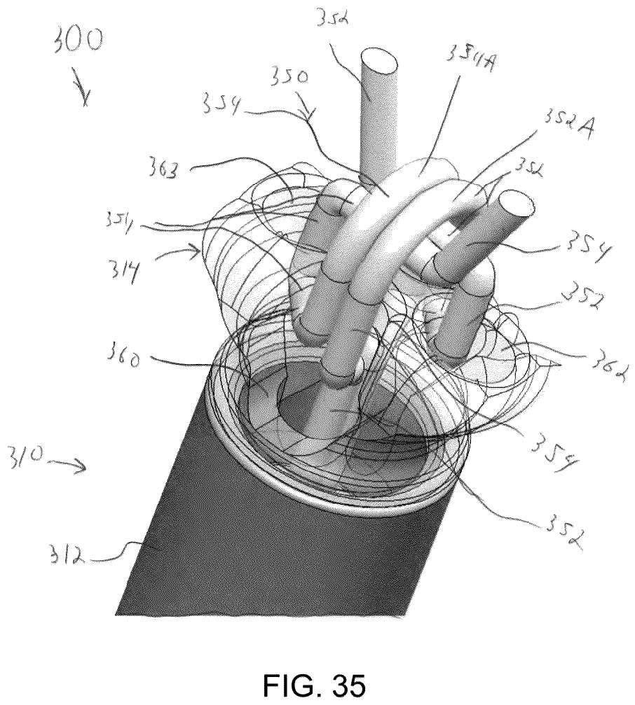

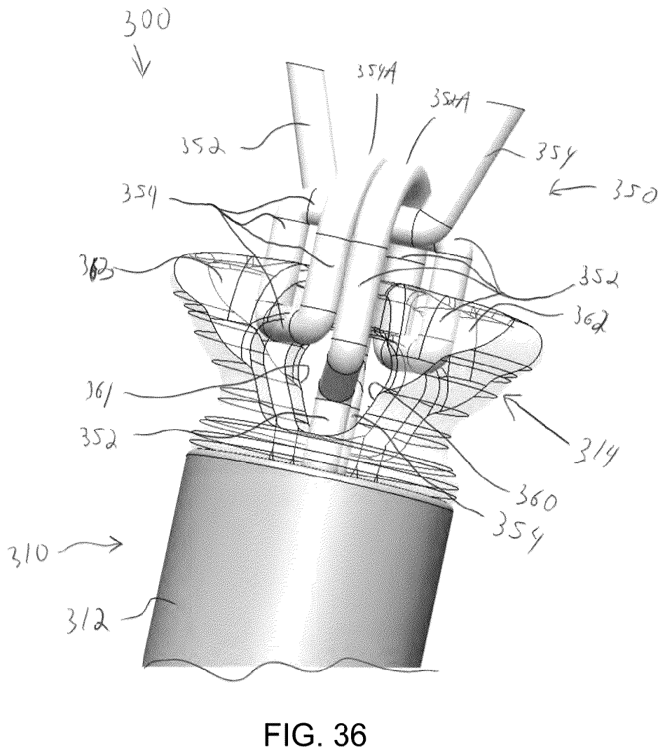

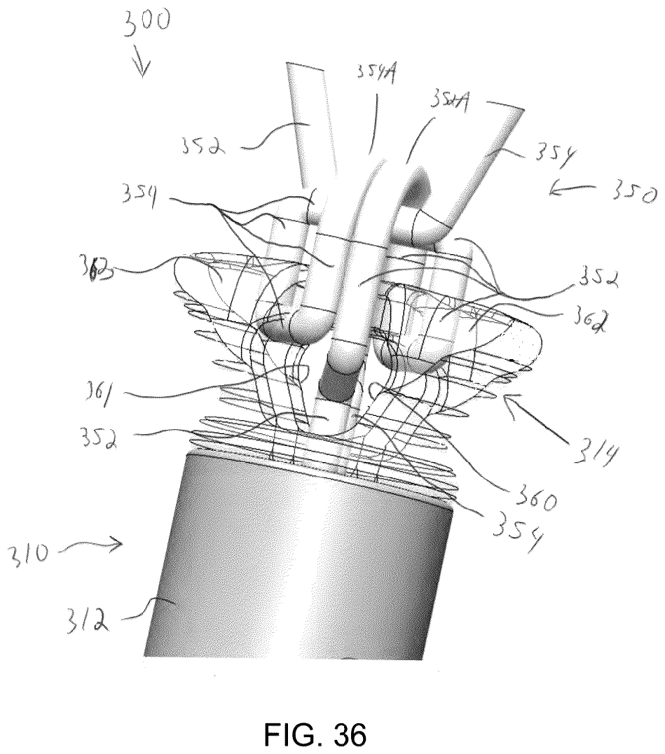

Another exemplary stabilization implant system 300 is shown in FIGS. 29-39. The stabilization implant system 300 of FIGS. 29-39 is substantially similar to the stabilization implant system 300 described above with respect to FIGS. 1-18, and therefore like reference numerals preceded with “3” as opposed to “1” are used to indicate like components, aspects, functions, portions, members, functions and the like, and the description above directed to thereto equally applies, and may not be repeated hereinbelow for brevity and/or clarity purposes.

The implant system 300 may be, for example, configured to heal syndesmotic ligaments post-operatively and/or stabilize syndesmotic joint, such as but not limited to a distal tibiofibular syndesmosis. The implant system 300 is configured to selectively constrain motion between two or more syndesmotic bones (e.g., in all directions) to allow for one or more syndesmotic ligaments extending therebetween to heal and/or to stabilize the syndesmotic joint. The implant system 300 allows for only a limited amount of relative movement/motion between bones in which it is implanted or installed, and such relative movement/motion and/or forces acting between the bones is be controlled (e.g., resisted/inhibited and/or encouraged/enhanced).

The components and portions of the implant system 300 may be made of, for example, titanium, stainless steel, polymers, polyester, polypropylene, UHMWPE, thermoplastic (e.g., thermoplastic urethane), bio-resorbable materials or any other biocompatible material so that the implant system 300 is configured to be implanted into a mammalian patient, such as a human patient.

As shown in FIGS. 29-39, the implant system 300 provides for allow dynamic stabilization/fixation of a joint between two bones (either naturally distinct bones or bone portions/segments), such as bones of a syndesmotic joint (e.g., a distal fibula and tibia of a distal tibiofibular syndesmosis). The implant system 300 is configured to provide dynamic stabilization through constrained and/or controlled relative motion between the bones. The area of allowed constrained controlled motion provided by the implant system 300 may be set in a space or gap between adjacent bones or bone segments (such as the syndesmotic joint/space between the distal the fibula and tibia).

As shown in FIGS. 29-37 and 39, the implant system 300 includes a flexible suture or tether 350 as a constraint and/or tension member that extends through portions of, and from, a dynamic bone anchor 310. As also shown in FIGS. FIGS. 29-37 and 39, a medial portion 356 of the tether 350 extending from the dynamic bone anchor 310 passes through, or is otherwise coupled with, a bone anchor 330. The dynamic bone anchor 310 and the bone anchor 330 are configured to respectively be coupled to and be implanted at least partially within first and second bones, such as bones of a syndesmotic joint (e.g., a distal fibula and tibia, respectively, of a distal tibiofibular syndesmosis), with the tether 350 extending between and coupled to the dynamic bone anchor 310 and the bone anchor 330.

The bone anchor 330 may be coupled with an intermediate loop portion 356 of the tether 350 extending from the dynamic bone anchor 310, and a medial loop portion 358 of the intermediate loop portion 356 and first and second retention portions 352, 354 of the tether 350 being knotlessly coupled to the dynamic bone anchor 310 (which prevents the tether 350 from translating through the dynamic bone anchor 310 in a direction that enlarges the intermediate loop portion 356, as explained further below). As also explained below, the medial portion 358 of the intermediate loop portion 356 of the tether 350 is retained in the dynamic bone anchor 310 via an elastic or resilient member that provides dynamically controlled translation between the dynamic bone anchor 310 and the bone anchor 330. The implant system 300 thereby provides dynamic stabilization of the joint between the first and second bones.

As shown in FIGS. 29-31, 33 and 39, the intermediate loop portion 356 of the tether 350 may pass through the soft bone anchor 330 a plurality of times. For example, a first loop portion of the intermediate loop portion 356 extending from the first retention portion 352 (within the dynamic bone anchor 310) may extend through a portion of the soft bone anchor 330 to the medial loop portion 358, and a second loop portion of the intermediate loop portion 356 extending from the second retention portion 354 (within the dynamic bone anchor 310) may extend through a second portion of the soft bone anchor 330 to the medial loop portion 358. The first and second portions of the soft bone anchor 330 may be the substantially same portions of the soft bone anchor 330, or the portions may longitudinally/axially overlap or be spaced from each other.

In some embodiments, the first and second loop portions of the intermediate loop portion 356 of the tether 350 may extend through the soft bone anchor 330 to the medial loop portion 358 in differing longitudinal/axial directions. For example, as shown in FIGS. 29-31, 33 and 39, the first and loop portion may extend from the first retention portion 352 and into a first longitudinal/axial portion of the soft bone anchor 330, and through the soft bone anchor 330 to a second longitudinal/axial portion of the soft bone anchor 330, and the second and loop portion may extend from the second retention portion 354 and into a third longitudinal/axial portion of the soft bone anchor 330 that is proximate to the second longitudinal/axial portion and distal to the first longitudinal/axial portion, and through the soft bone anchor 330 to a fourth longitudinal/axial portion of the soft bone anchor 330 that is proximate to the first longitudinal/axial portion and distal to the second longitudinal/axial portion. In this way, the first and second and loop portions may overlap as they extend longitudinally/axially within the soft bone anchor 330 (such as within an inner longitudinal and/or elongated passageway or cavity thereof).

In some embodiments, the first and fourth portions of the soft bone anchor 330 are proximate to a first longitudinal/axial end of the soft anchor 330 and distal to a second longitudinal/axial end of the soft anchor 330, and the second and third portions of the soft bone anchor 330 are proximate to the second longitudinal/axial end and distal to a first longitudinal/axial end. In some embodiments, the first and fourth portions of the soft anchor 330 are the same portion of the soft anchor 330 or are positioned substantially adjacent to each other, and the second and third portions of the soft anchor 330 are the same portion of the soft anchor 330 or are positioned substantially adjacent to each other. As explained further below, the medial loop portion 358 extending between the first and second and loop portions may extend into the dynamic bone anchor 310 and extend/be looped over/through a tether post 320 that is translatably resiliently retained within a cavity/lumen/cannulation of an anchor body 312 of the dynamic bone anchor 310. Movement of the tether post 320 within the anchor body 312 thereby either lengthens or shortens the size/length of the intermediate loop portion 356 to adjust the distance between the soft bone anchor 330 and the dynamic bone anchor 310 (as the tether 150 slides through the soft bone anchor 330). It is noted that the tether 350 may be slidably coupled with/through the soft bone anchor 330 to allow the soft bone anchor 330 to slide along the intermediate loop portion 356 of the tether 350 as it adjusts in size/length.

The tether 350 may be configured to mimic the function, location and/or length of an interosseous ligament, for example. The tether 350 may be of any thin, long and substantially freely manually flexible configuration or structure, such as a suture, strand, cable or string-like configuration. The tether 350 may be, for example, a biomedical suture or tether (e.g., a stranded cerclage cable), or similar construct. In some embodiments, the tether 350 may be formed of, for example, polymer, polyester, polypropylene or UHMWPE suture, strands or filaments, braids thereof, or a like material, as known by one of ordinary skill in the art. The tether 350 may be, for example, a suture (e.g., a braided suture), such as a single cross-section strand of suture or multiple loops of suture. For example, the tether 350 may be a UHMWPE and polypropylene co-braid suture.

The soft deformable anchor 330 may be solid or porous. For example, the soft deformable anchor 330 may be molded via a solid material. As another example, the soft deformable anchor 330 may be woven from one or more threads, fibers or filaments. In some embodiments, the soft deformable anchor 330 may be formed via one or more machining, additive and/or extrusion formation process. The soft deformable anchor 330 may be of one-piece construction or formed of a plurality of members or portions (and/or materials). The soft deformable anchor 330 may be formed or made from any biocompatible material(s). For example, the soft deformable anchor 330 may be made or formed of polyester, polyethylene (e.g., ultra-high-molecular-weight polyethylene), polypropylene, nylon (e.g., poylamide), silk, polyglycolic acid (PGA), polydioxanone (PDO), polylactic acid (PLA), polylactic-co-glycolic acid (PLGA), stainless steel, or a combination thereof. In one example, the soft deformable anchor 330 is formed of polyester.

The tether 350 may or may not be elastically axially/longitudinally stretchable or deformable. In some embodiments, the tether 350 may have one or more openings or gaps along its length that is configured to allow another tether or suture (or the like) to pass therethrough. In this way, the tether 350 my act as a tether or suture passer to translate another tether or suture to and/or through at least a portion of the implant system 300.

As explained above, a portion of the intermediate or medial loop portion 358 of the tether 350 extends within the bone anchor 330. The bone anchor 330 can be a soft deformable bone anchor, such as an all-soft suture anchor (ASAs). For example, the bone anchor 330 may be a tube, sleeve or tape made of suture or tether material (e.g., woven) through which the tether 350 is woven. In some other embodiments, the tube, sleeve or tape may be solid and/or formed of a resilient material, such as an elastomer.

The soft deformable anchor 330 may be elongated along a longitudinal direction in a neutral state. In some embodiments, the soft deformable anchor 330 may define a longitudinal length within the range of 5 mm and 35 mm. The soft deformable anchor 330 may be a size proportion bigger than the tether 350. For example, the width or diameter of the soft deformable anchor 330 may be larger than that of the tether 350.

In some embodiments, the soft deformable anchor 330 may be formed of an annular side wall that defines an inner cavity and first and second longitudinal ends. The soft deformable anchor 330 may thereby be hollow. The size of the inner cavity may be larger than the tether 350 such that the tether 350 is able to pass though the inner cavity. The inner cavity may extend through the soft deformable anchor 330 such that the inner cavity is open or accessible at one or both of the longitudinal ends, or the longitudinal ends may be closed or sealed. In some embodiments, the soft deformable anchor 330 may not define or include an inner cavity.

The soft deformable anchor 330 is flexible, collapsible, deformable, bendable, stretchable or otherwise re-arrangeable in overall shape. In a natural, neutral or non-deformed state, as shown in FIGS. 29-33 and 39, the soft deformable bone anchor 330 may extend substantially linearly or generally arcuately along the longitudinal direction. The soft deformable bone anchor 330 is configured such that tension on the portions on the suture extending from the soft deformable anchor 330 in a lateral direction that is angled from the longitudinal direction (e.g., substantially perpendicularly from the outer side surface of the anchor 330) deforms the soft deformable anchor 330 into a curved, bent-over, compressed or “bunched” shape, as discussed above with respect to the anchor 130.

The tether 350 (e.g., the intermediate loop portion 356, including the medial loop portion 358 thereof) may extend through a side or portion of the soft deformable anchor 330 one or more times which, when tension in the lateral direction (at least generally), deforms the anchor 330 into a dense concave/convex, arcuate, folded, “C” or “U” and/or “V” shape. For example, in an installed or neutral state, the anchor 330 may be bent and/or pulled together (e.g., via the tether 350) such that it assumes a sharply arcuate (e.g., defined by a relatively small radius) U-shape or V-shape. The tether 350 (e.g., the intermediate loop portion 356, including the medial loop portion 358 thereof) may be tensioned laterally such that the tether 350 deforms, compresses or pulls together a side or portion of the soft deformable anchor 330 such that the deformable anchor 330 or a portion thereof bunches up, balls up or otherwise becomes more densely arranged in the sharply arcuate shape such that it defines a larger width, lateral/cross-sectional dimension, or other size. In this way, the soft deformable bone anchor 330 is configured such that when it is inserted into a properly sized/shaped hole, tunnel or cavity in a bone and the suture portions extending from the anchor 330 (e.g., the intermediate loop portion 356, including the medial loop portion 358 thereof) are tensioned/pulled at least generally laterally, the anchor 330 is cinched up and enlarged in at least one dimension such that the anchor 330 compress against the side wall of the hole/cavity in the bone, creating a dense “ball” that is squeezed within the hole/tunnel/cavity and, thereby, anchored therein. As explained further below, in some exemplary methods, for dynamic stabilization of a distal tibiofibular syndesmosis, a hole/tunnel/cavity can be formed in the distal tibia (which may be formed via drilling the distal tibia). The anchor 330 may thereby be a tibial anchor. It is noted that the configuration of the anchor 330 allows it to be placed in a bone hole/tunnel/cavity with a substantially smaller diameter (e.g., 1-3 mm), thereby preserving bone and allowing for more bone preservation, for example.

As noted above and shown in FIGS. 29-39, the implant system 300 includes the dynamic bone anchor 310. The medial loop portion 358 of the intermediate loop portion 356 (of the flexible suture or tether 350), and the first and second retention portions 352, 354 extending to the intermediate loop portion 356, extends in/through dynamic bone anchor 310. As discussed above, the intermediate loop portion 356 extending from the dynamic bone anchor 310 passes through, or is otherwise coupled with, the bone anchor 330. As also noted above, the dynamic bone anchor 310 is configured to be coupled to and implanted at least partially within a second bone, such as a bone of a syndesmotic joint (e.g., a distal fibula of a distal tibiofibular syndesmosis), with the tether 350 (the first and second retention portions 352, 354 and the intermediate loop portion 356 (including the medial loop portion 358 thereof)) extending between and coupled to the dynamic bone anchor 310 and the bone anchor 330.

With reference to in FIGS. 29-39, in some embodiments, the dynamic bone anchor 310 includes an anchor body 312 and a head portion 314. The anchor body 312 may be positioned at and/or define a first longitudinal/axial end or end portion of the bone anchor 310, and the head portion 314 may be positioned at and/or define a second longitudinal/axial end or end portion of the bone anchor 310. In some embodiments, the dynamic bone anchor 310, as a whole, may be substantially or generally cylindrical.

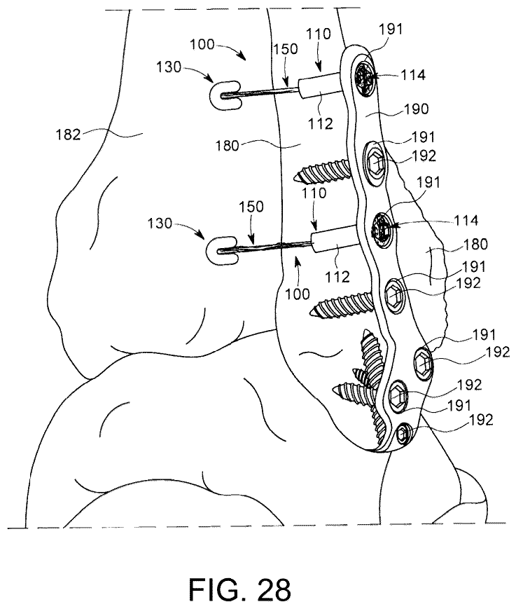

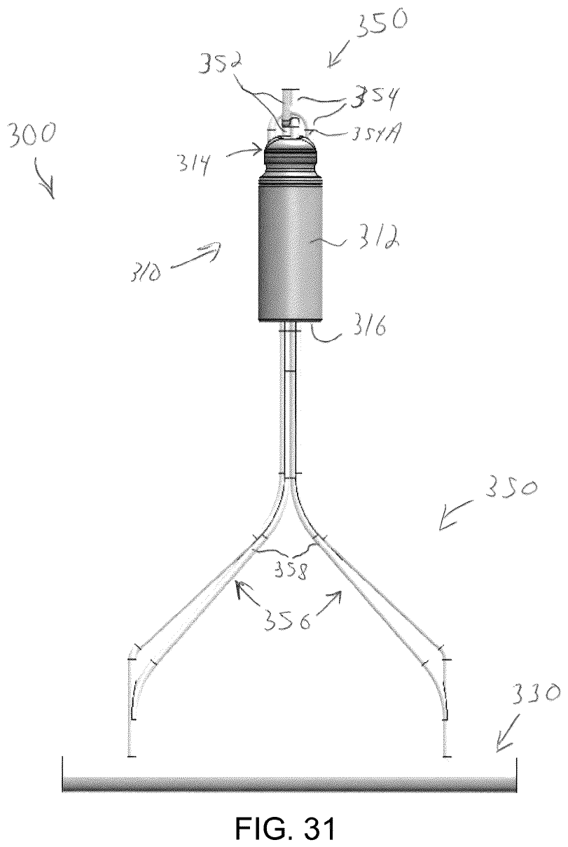

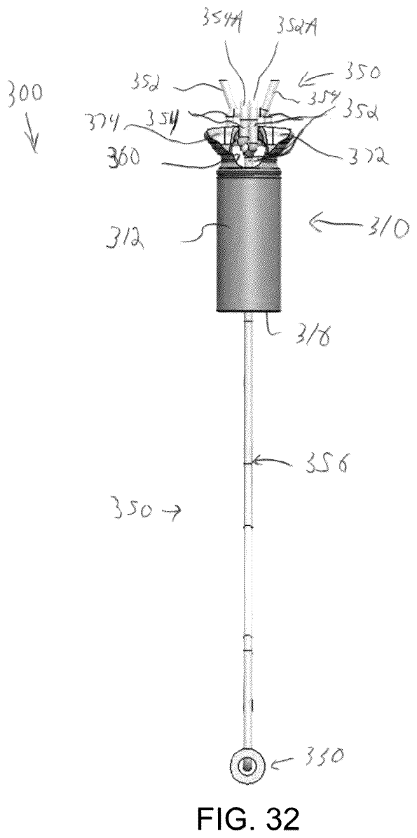

As shown in FIGS. 630, 33-35, 37 and 38, the anchor body 312 may include an inner cavity/lumen/cannulation/opening that extends longitudinally/axially therethrough. The inner cavity of the anchor body 312 may be open at the first end of the bone anchor 310 via a tether aperture 315 in an end wall 316 of the anchor body 312. The tether aperture 315 is configured to allow the medial loop portion 358 of the intermediate loop portion 356 and the first and second retention portions 352, 354 of the tether 350 to extend therethrough, as shown in FIGS. 30 and 34.

As shown in FIG. 34, the anchor body 312 contains a resiliently/elastically deformable bumper 318 and a tether post 320 movably contained within the inner cavity thereof as the bumper 318 is larger than the tether aperture 315. The tether post 320 is at least axially/longitudinally translatable within the inner cavity of the anchor body 312. The resilient member or bumper 318 is at least axially/longitudinally resiliently compressible and/or elongatable within the inner cavity of the anchor body 312. The bumper 318 may thereby be considered at least axially/longitudinally translatable within the inner cavity as at least some portions of the bumper 318 translate axially/longitudinally within the inner cavity as it axially/longitudinally compresses/shortens and elongates. The bumper 318 and/or the tether post 320 may also be rotatably retained within the inner cavity of the anchor body 312.

The bumper 318 is axially/longitudinally positioned and trapped between the end wall 316 of the anchor body 312 and the tether post 320, and thus is axially/longitudinally resiliently/elastically compressible and/or elongatable therebetween. The tether post 320 may abut an axial/longitudinal end of the bumper 318, and the other axial/longitudinal end of the bumper 318 may abut the end wall 316. As shown in FIG. 34, the bumper 318 may have a cavity/lumen/cannulation/opening that extends longitudinally/axially therethrough. The cannulation of the bumper 318 is configured to allow the intermediate loop portion 356 and the first and second retention portions 352, 354 of the tether 350 to extend axially/longitudinally therethrough and to and past, respectively, the tether post 320.

In some embodiments, the bumper 318 may be an elastic or resilient spring-like member. The bumper 318 may be comprised of one or more elastically deformable member or material. For example, the bumper 318 may be one or more springs (e.g., disc or coil spring) or elastically compressible disc or tube, or a combination thereof. For example, the bumper 318 may comprise an elastically compressible disc (e.g., elastomeric, polymer, polyurethane or polyethylene disc), tube (e.g., a polyurethane tube) or coil spring. In some embodiments, the bumper 318 comprises at least one urethane tube or like member, such as at least one polycarbonate urethane (PCU) tube or like member or at least one thermoplastic polyurethane (TPU) tube or like member.

The bumper 318 is configured such that it elastically deforms to tension the medial loop portion 358 of the tether 350 that pulls the anchor portion 330 and the dynamic anchor 310 together, and/or allows a limited degree of relative movement between the anchor portion 330 and the dynamic anchor 310. The tether post 320 may be forced, variably, axially/longitudinally toward the end wall 316 of the anchor body 312 via tension on the medial loop portion 358 of the intermediate loop portion 356 of the tether 350 such that it is variably compresses the bumper 318 between the tether post 320 and the end wall 316 of the anchor body 312.

As shown in FIG. 34, the tether post 320 may have a cavity/lumen/cannulation/opening that extends longitudinally/axially therethrough. The cannulation of the tether post 320 is configured to allow the first and second retention portions 352, 354 of the tether 350 to extend axially/longitudinally therethrough, through/past the bumper 318, and to the intermediate loop portion 356. The cannulation of the tether post 320 also allows the medial loop portion 358 to extend partially therethrough and over a post portion of the tether post 320. The intermediate loop portion 356 may thereby be looped over the post portion of the tether post 320, and the post portion may prevent the medial loop portion 358 from translating or otherwise decoupling from the dynamic anchor 310 when tensioned.

As shown in FIGS. 29-39, the head portion 314 may be configured to mate with the anchor body 312 (or they may be one piece), and includes a cavity/lumen/cannulation/opening that extends longitudinally/axially therethrough that allows the first and second retention portions 352, 354 of the tether 350 to extend therethrough. The cannulation of the head portion 314 may be configured to allow the first and second retention portions 352, 354 to be retained, coupled, affixed or the like to the head portion 314 and each other such that the first and second retention portions 352, 354 are prevented from sliding or otherwise translating back through the dynamic anchor 310 to elongate/enlarge the intermediate loop portion 356 of the tether 350, and thereby the medial loop portion 358 and the distance between the dynamic anchor 310 and the soft anchor 330.

As shown in FIGS. 33-38, the head portion 314 may include a plurality of apertures that are in communication with the cannulation configured such that the first and second retention portions 352, 354 can extend through and around portions of the head portion 314. In some such embodiments, the first and second retention portions 352, 354 extend through and around portions of the head portion 314 in a knotless arrangement that adjustably secures or fixes the first and second retention portions 352, 354. For example, as shown in FIGS. 33-38, the head portion 314 may include a first aperture 360, a second aperture 361, a third aperture 362 and a fourth aperture 363. In the illustrated exemplary embodiment, the first retention portion 352 of the tether 350 extends through the first aperture 360 from the cannulation of the head portion 314, over the top side of the head portion 314 and down into the second aperture 361, and then back up through the third aperture 362, as shown in FIGS. 33-38. Similarly, the second retention portion 354 of the tether 350 extends through the second aperture 361 from the cannulation of the head portion 314, over the top side of the head portion 314 and down into the first aperture 360, and then back up through the fourth aperture 363, as shown in FIGS. 33-38. The pathway of the first and second retention portions 352, 354 thereby each form an adjustment loop 352A, 354A above the top side of the head portion 314. As also shown in FIGS. 33-38, the end portions of the first and second retention portions 352, 354 may pass under one or both of the adjustment loops 352A, 354A. In this way, when the tether 350 is tensioned, the adjustment loops 352A, 354A are pulled down onto/towards the top side of the head portion 314 and compress against end portions of the first and second retention portions 352, 354 to fix the tether 350 without the use of a knot.

The first and second retention portions 352, 354 may thus each extend through the head portion 314 past the outer top side thereof more than once (e.g., twice). As noted above, the first and second retention portions 352, 354 of the tether 350 comprise opposing end portions of the tether 350. The first retention portion 352 thus forms the first loop portion 352A extending from the head portion 314 past the outer top side thereof, and a first end portion of the first retention portion 352 extends from the head portion 314 past the outer top side (from the third aperture 362). Likewise, the second retention portion 354 forms the second loop portion 354A extending from the head portion 314 past the outer top side thereof, and a second end portion of the second retention portion 354 extends from the head portion 314 past the outer top side (from the fourth aperture 363). As discussed above, the first end portion of the first retention portion 352 extends over the outer top side and through the first loop portion 352A and the second loop portion 354A, and the second end portion of the first retention portion 354 extends over the outer top side and through the first loop portion 352A and the second loop portion 354A to adjustably knotlessly couple the first and second retention portions 352, 354 to the head portion 314 of the dynamic bone anchor 310.

As shown in FIGS. 33-38, the first and second apertures 360, 361 may be open at respective lateral sides of the head portion 314. In contrast, as also shown, the third and fourth apertures 362, 363 may not be exposed at the lateral sides of the head portion 314, but rather comprise openings at the top side of the head portion 314 formed in the top surface portion of the head portion 314.

The back or underside of the head portion 314 of the dynamic anchor 310 may be configured to mate with bone and/or a bone plate. For example, as shown in FIGS, 33-39, the head portion 314 may include an underside or backside taper that is configured to engage bone and/or a countersunk aperture of a bone plate, for example. In some other embodiments, the back or underside of the head portion 314 may include threads configured to engage bone and/or a threaded aperture of a bone plate, for example. The outer surface of the anchor body 312 may likewise be configured substantially smooth or with threads, for example.

As shown in FIGS, 33-39, the enlarged top portion of the head portion 314 may comprise an oblong cross-sectional shape, which may allow it to seat on bone and/or a bone plate at an angle without a pronounced raised portion of the head portion 314. The enlarged top portion of the head portion 314 may compress first and second lateral side portions 372, 374 that extend laterally past a base portion of the head portion 314. The lateral side portions 372, 374 may comprise a tapered underside. Third and fourth lateral side portions of the head portion 314 may extend between the first and second lateral side portions 372, 374. The third and fourth lateral side portions may not extend laterally past the base portion, and may comprise at least a portion of the third and fourth apertures 362, 363, as shown in FIGS, 33-39.

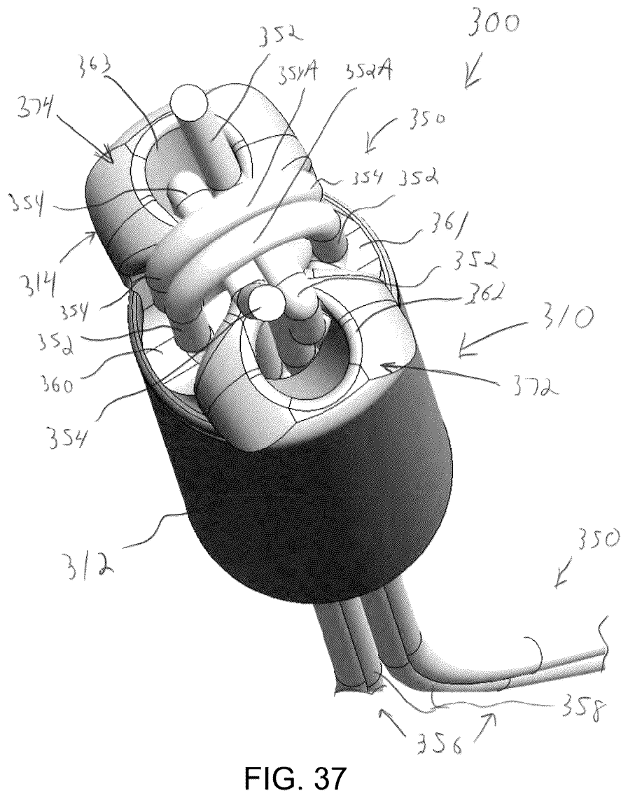

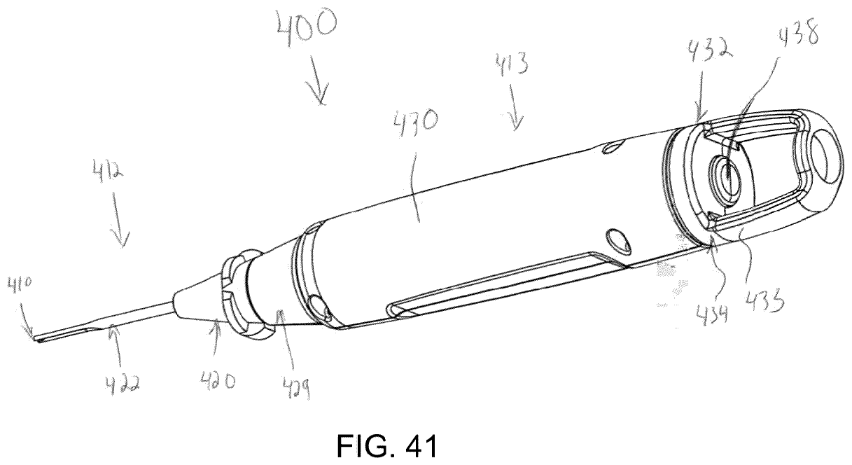

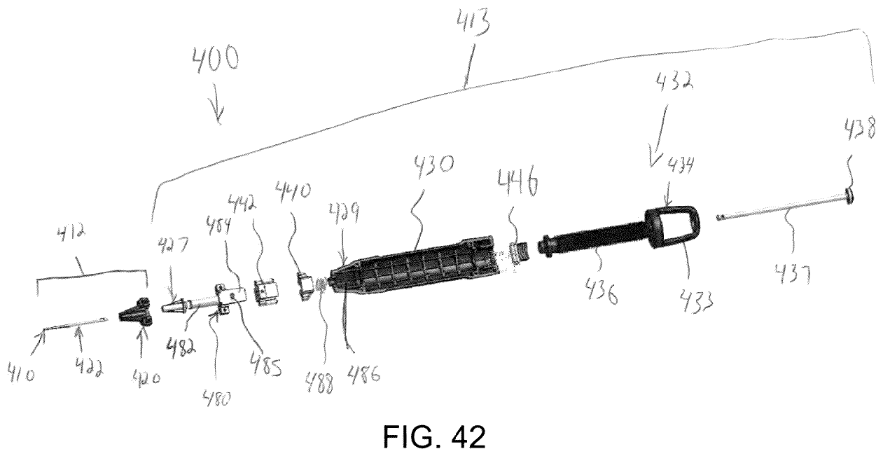

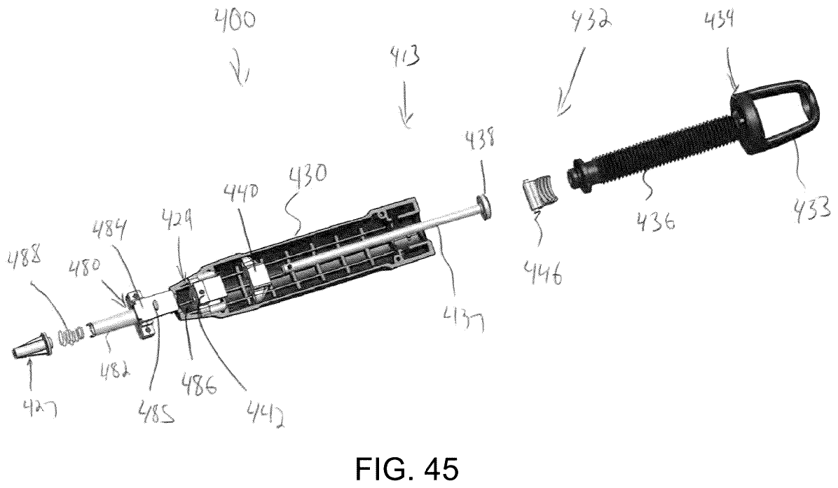

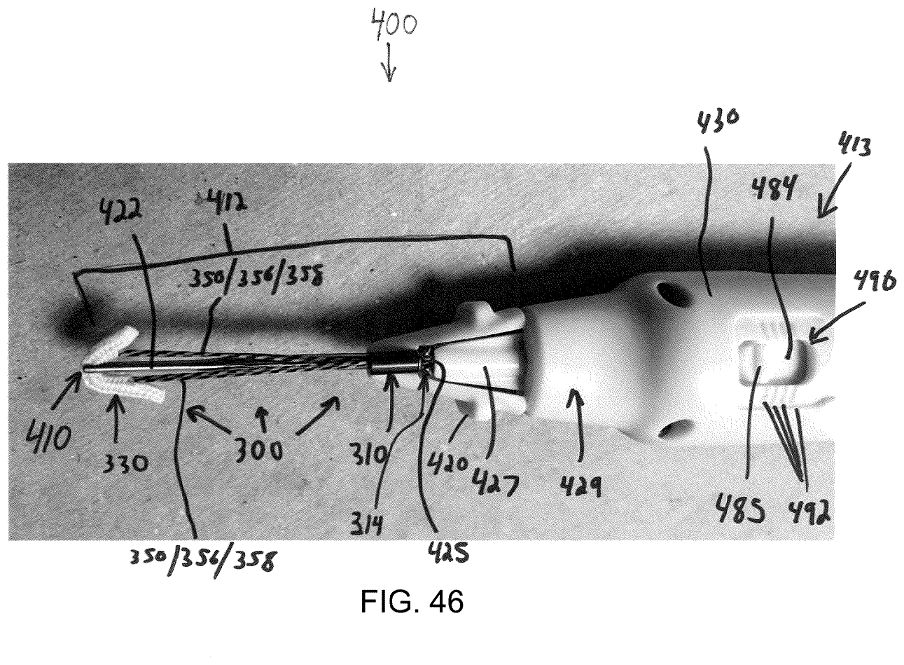

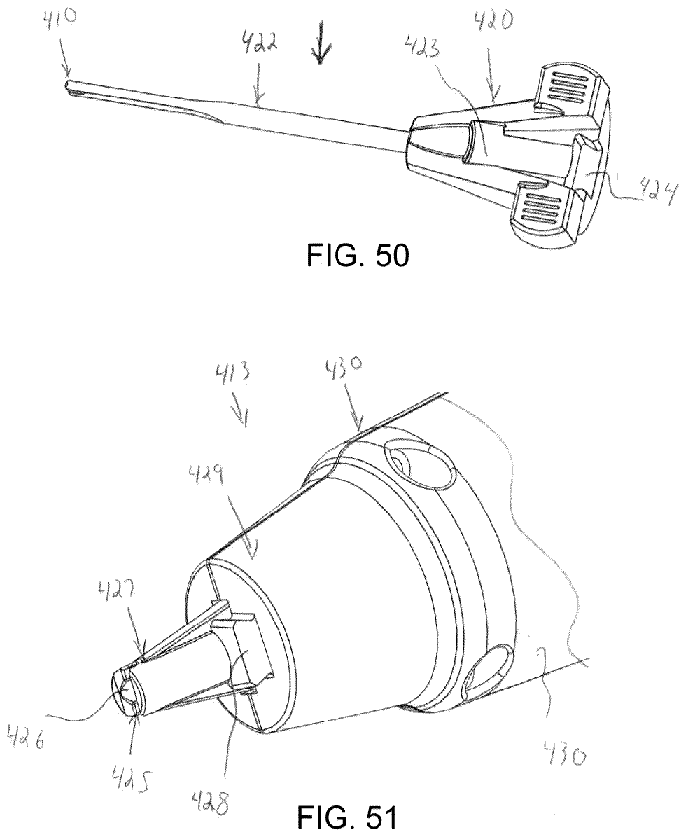

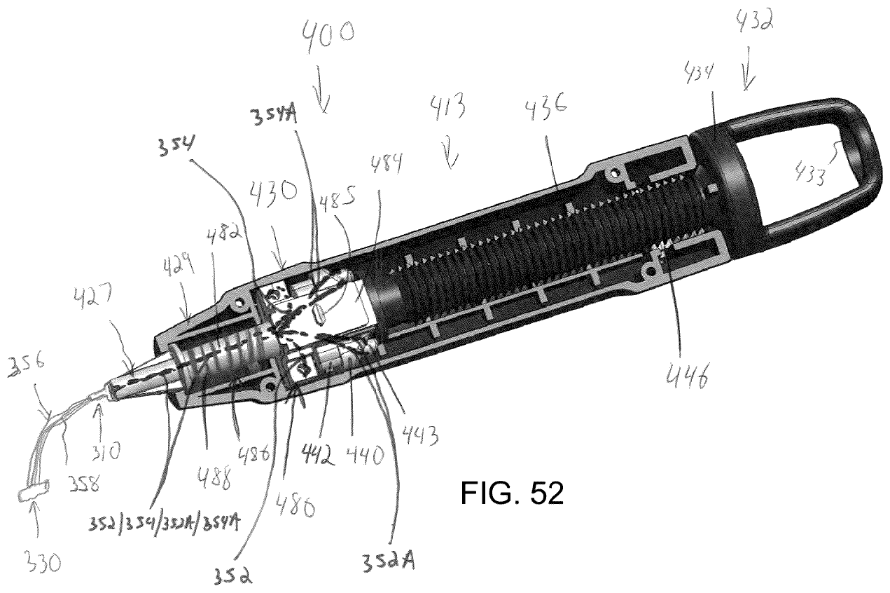

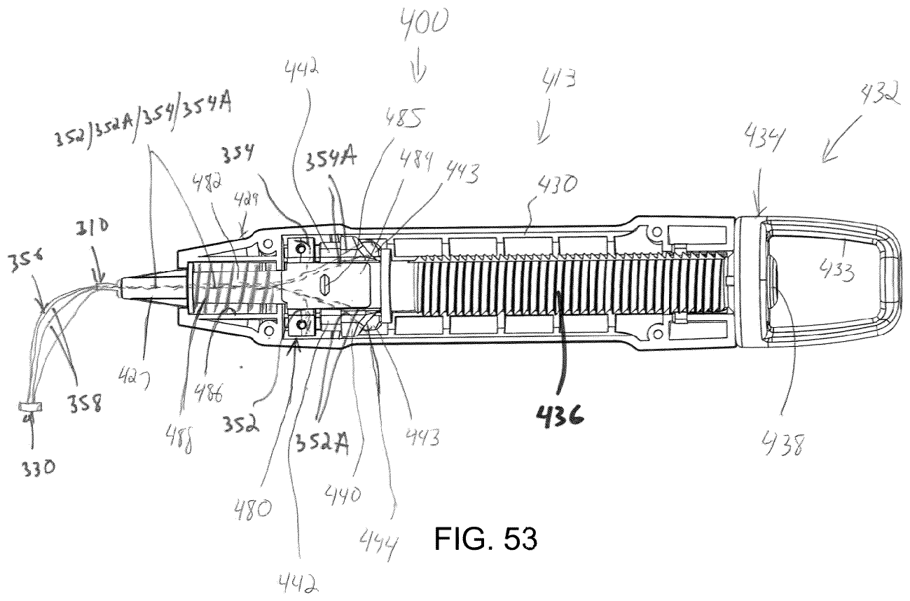

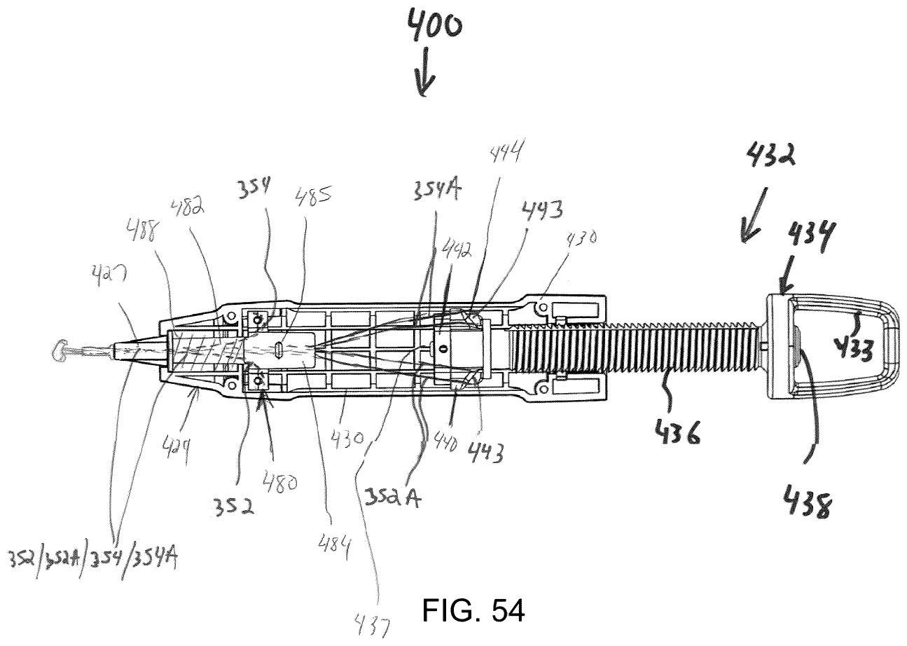

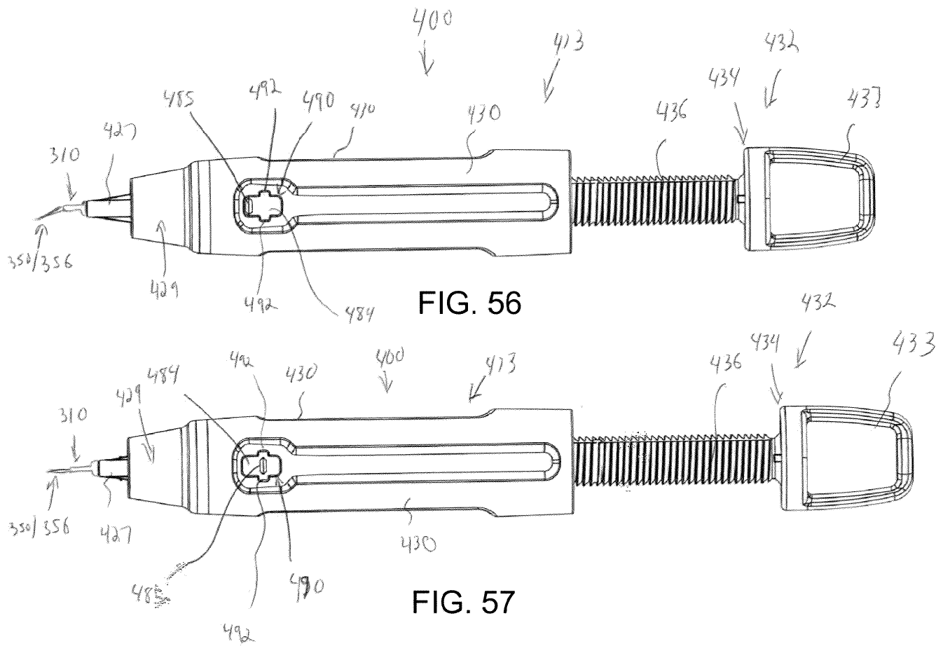

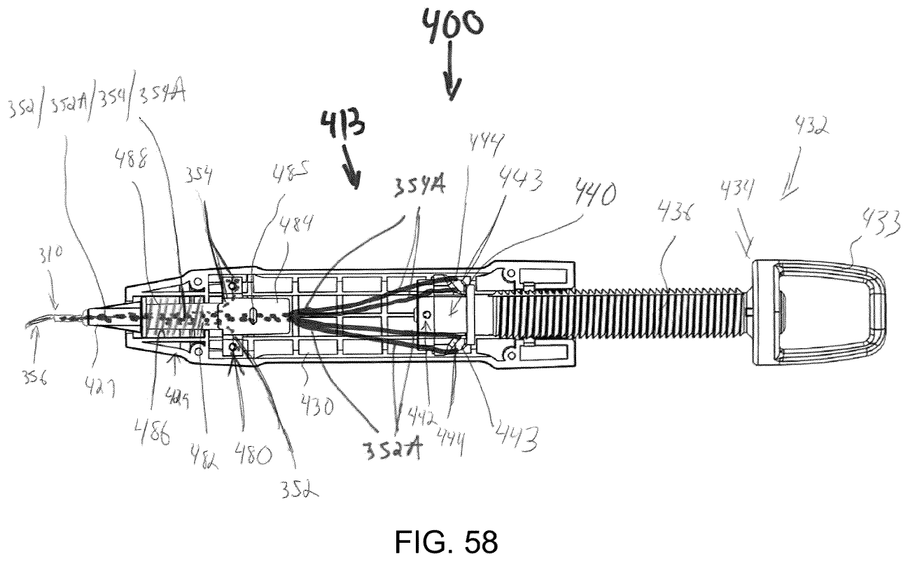

Another exemplary implant insertion and tensioning instrument 400 is shown in FIGS. 40-62. The insertion and tensioning instrument 400 of FIGS. 40-62 is similar to the insertion and tensioning instrument 200 described above with respect to FIGS. 19-27, and therefore like reference numerals preceded with “4” as opposed to “2” are used to indicate like components, aspects, functions, portions, members, functions and the like, and the description above directed to thereto equally applies, and may not be repeated hereinbelow for brevity and/or clarity purposes.

The implant insertion and tensioning instrument 400 is configured to implant, reduce and tension a stabilization implant system as described herein, such as the stabilization implant system 100 or the stabilization implant system 300 described above. For illustration and/or explanatory purposes, the implant insertion and tensioning instrument 400 is shown and described hereinbelow in use with the stabilization implant system 300 if FIGS. 29-39. However, it is specifically contemplated that the stabilization implant system 100, or another the stabilization implant system that is like the systems 100 and 300, may equally be employed.

The implant insertion and tensioning instrument 400 is configured to implant, reduce and tension the stabilization implant system 300, across a joint between two bones (either naturally distinct bones or bone portions/segments), such as bones of a syndesmotic joint (i.e., syndesmotic bones) (e.g., a distal fibula and tibia of a distal tibiofibular syndesmosis) so that the implant system 300 provides dynamic stabilization through constrained and/or controlled relative motion between the syndesmotic bones (and thereby repair and/or allow for one or more syndesmotic ligaments extending therebetween to heal and/or to stabilize the syndesmotic joint). The implant insertion and tensioning instrument 400 and the stabilization implant system 300 (and/or another stabilization implant system) may cooperatively form a system, such as an implant system for dynamic stabilization of a syndesmosis joint.

As shown in FIGS. 40, 41 and 46-62, the instrument 400 comprises a first portion 412 that is removably coupled to a second portion 413. The first portion 412 may extend longitudinally from a front end or tip portion of the second portion 413, and the first and second portions 412, 413 may be longitudinally fixed together when removably coupled together. As discussed further below, the first and second portions 412, 413 may be configured such that they are decoupled by relative lateral movement therebetween along at least a first lateral direction.

As shown in FIGS, 46 and 47, and discussed further herein, the soft bone anchor 330 may be removably retained on a free end portion 410 of the first portion 412 of the instrument 400, and the dynamic bone anchor 310 may be removably retained on at least one of the first portion 412 and the second portion 413 of the instrument 400.

As shown in FIGS. 40-43 and 46-50, the first portion 412 may be or form a soft bone anchor inserter or inserter portion configured to removably retain the soft bone anchor 330, and pass/implant the soft bone anchor 330 through, into and/or onto one or more bones or bone cavities. For example, as described herein, the first portion 412 may be configured to retain the soft bone anchor 330 thereon, insert/pass the soft bone anchor 330 through a hole/tunnel extending through at least a portion of two or more bones (e.g., a distal tibia and fibula), and implant the soft bone anchor 330 in or against an outer surface of one of the bones (e.g., a tibia). After implantation of the soft bone anchor 330, the soft bone anchor 330 may be decoupled/removed from the first portion 412, and the first portion 412 can be decoupled from the second portion 413.

As shown in FIGS. 40-43 and 46-50, the first portion 412 may comprise a base portion 420 that is removably coupled to a tip portion of the second portion 413, and a longitudinally elongated inserter portion 422 extending longitudinally from the base portion to a free end 410 thereof. The inserter portion 422 may be configured as a stiff elongate rod or like member such that it is able to be inserted into, and extend in/through, a hole in one or more bones, such as a drilled hole.

As shown in FIGS. 40-43 and 46-50, the free end 410 of the inserter portion 422 may be forked. For example, the free end 410 of the insertion portion 422 may have a pair of longitudinally extending (e.g., elongate) tines and a base retention portion extending therebetween. In some embodiments, the free end 410 may also include a pair of grooves extending longitudinally proximally from the base retention portion toward the base portion 420.

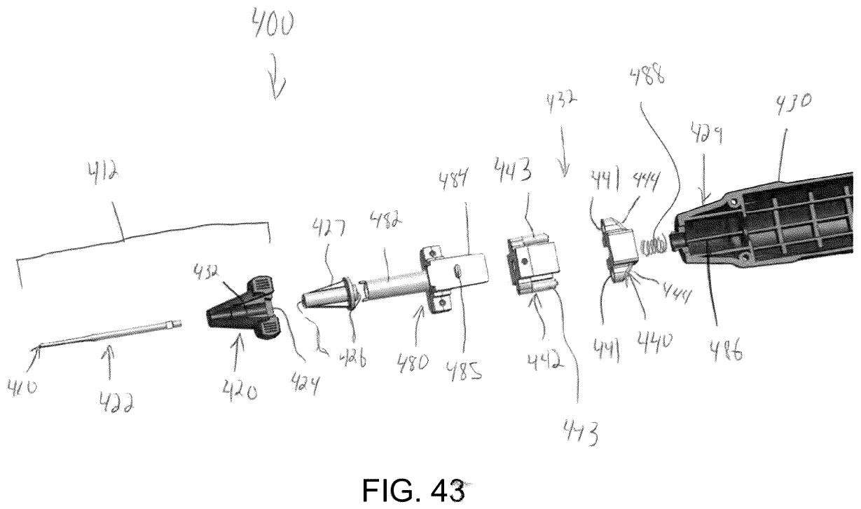

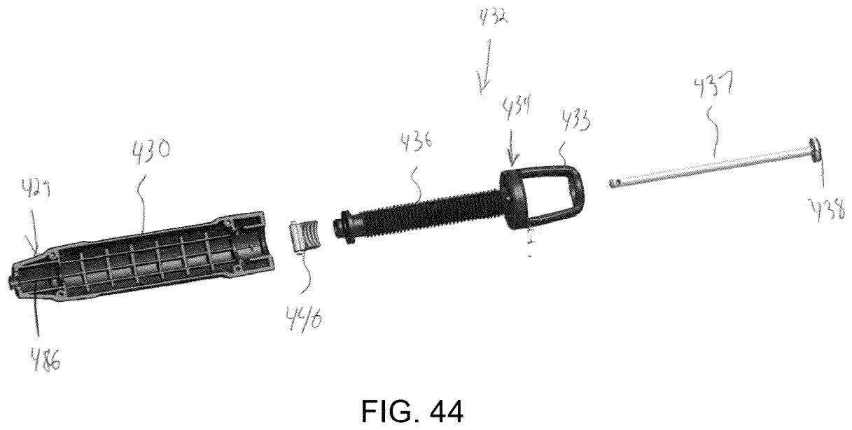

As shown in FIGS. 46 and 47, in a preinstalled, preconfigured and/or loaded state/arrangement/configuration of the implant system comprising the instrument 400 and the implant 300, the soft anchor 130 be removably retained or coupled on/over a forked free end 210 of the first insertion portion 212. The soft anchor 130 may be removably retained on the forked free end 210 via tension of the tether 150, which may be removably coupled to a second portion 213 of the instrument 200, as discussed further below. The soft anchor 130 is retained on the forked free end 210 such that end portions of the soft anchor 130 extends over or within the pair of grooves, and a medial portion of the soft anchor 130 extends over the base portion between the pair of tines.

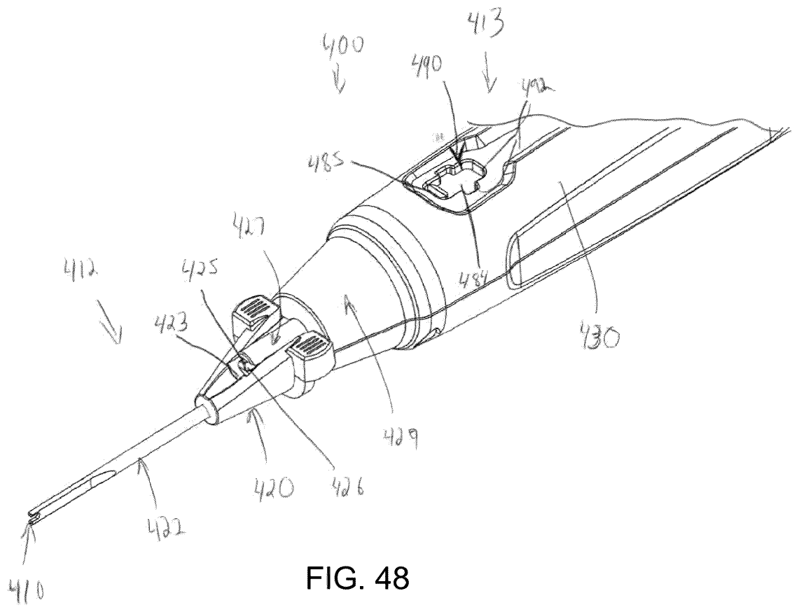

The instrument 400, and in particular, the first portion 412, may be utilized to introduce the soft anchor 330 into a cavity or hole in a first bone (e.g., lateral cortex of tibia) through a second bone (fibula). After insertion, the instrument 400 may be pulled back to tension the intermediate loop portion 356 and deform the soft anchor 330 to fix the soft anchor 330 to/on the bone and remove the soft anchor 330 from the end portion 410 of the first portion 412. After such implantation, the first portion 412 can be removed or decoupled from the second portion 413, which also exposes or releases the dynamic anchor 410, as shown in FIGS. 51-59.

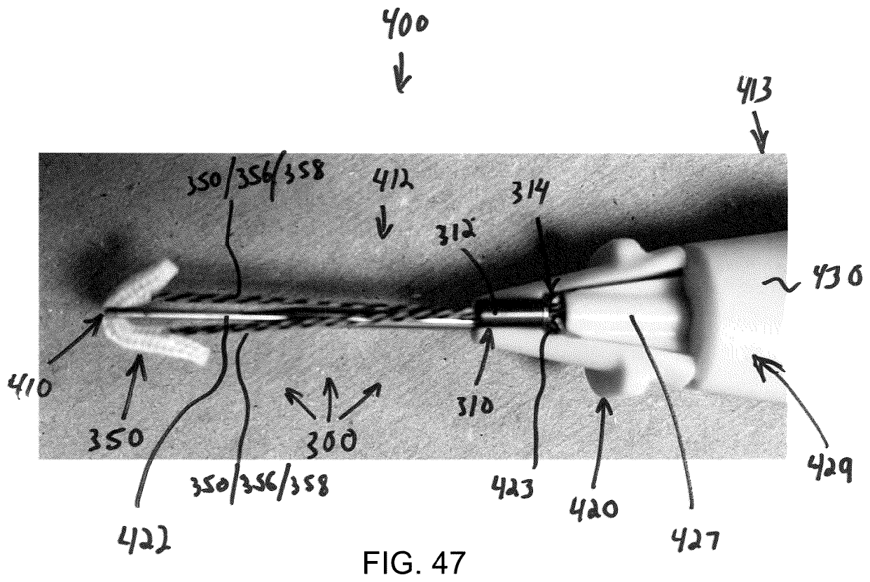

As shown in FIGS. 50 and 51, the first portion 412 may be removably coupled to the second portion 413 via a laterally arranged dovetail coupling configuration. For example, as shown in FIGS. 50 and 51, an inner lateral side of the base portion 420 of the first portion 412 may include a recess or mortise 424 that is configured to mate with a correspondingly/matingly shaped projection or tenon 428 on a lateral side of a tip portion 429 of the second portion 413 (or vice versa). The projection 428 may extend from a longitudinal end or wall of the tip portion 429. The recess 424 and projection 428 may be flared or widened as they extend longitudinally away from the tip portion 429 so that the first and second portions 412, 413 are longitudinally fixed together, but can be disassembled or decoupled via relative lateral movement therebetween.

The soft anchor 330 may be removably retained on the forked free end 410 via tension of the tether 350 (e.g., the intermediate portion 356), which may be removably coupled to a second portion 413 of the instrument 400, as discussed further below. The intermediate portion 356 of the tether 350 may thereby extend longitudinally from the soft bone anchor 330 at the free end 410 of the inserter portion 422 along the inserter portion 422 to the dynamic bone anchor 310. As shown in FIGS. 46 and 47, in the preinstalled, preconfigured and/or loaded state/arrangement/configuration of the implant system, the dynamic bone anchor 310 may be retained on a recess or groove 425 of a front end tip portion of the second portion 413. For example, the head portion 314 of the dynamic bone anchor 310 may be positioned in or against a recess or groove 425 in a tip member 427 of the second portion 413, as shown in FIGS. 46-49 and 51. The recess 425 may thereby be shaped and otherwise configured to accept at least a portion of the head portion 314 of the dynamic bone anchor 310 therein.

The dynamic bone anchor 310 may be retained on/in the recess or groove 425 the second portion 413 via tension of the tether 350 and/or the first portion 412 (when the first portion 412 is coupled with the second portion 413). As shown in FIGS. 46-50, an inner lateral side portion 423 of the base portion 420 of the first portion 412 may extend partially laterally over the recess 425 and end of the tip member 427 of the second portion 413 (but longitudinally spaced therefrom) when the first portion 413 is removably coupled to the second portion 413. The inner lateral side portion 423 of the base portion 420 may comprise a recess or shaped surface configured to partially over the recess 425 and end of the tip member 427, and potentially engage a portion of the dynamic bone anchor 310. In this way, the inner lateral side portion 423 of the base portion 420 and the recess 425 and end of the tip member 427 may form a cavity, pocket or opening that retains, contains or traps a portion of the dynamic bone anchor 310, such as the head portion 314 thereof, to removably retain the dynamic bone anchor 310 on the instrument 400 (when the first portion 413 is removably coupled to the second portion 413). In some embodiments, a portion of the inner lateral side portion 423 may be configured to engage or abut a portion of the anchor body 312 of the dynamic bone anchor 310.

As shown in FIGS. 40-49 and 51-60, the second portion 413 of the instrument 400 further comprises a main housing portion 430 and a tension handle assembly or portion 432 manually movably coupled with the main housing 430. The main housing 430 may form an internal cavity in which other components of the instrument 400 are housed, such as some of the components of the tension handle portion 432. The main housing 430 may be longitudinally elongated, with the tip member 427 positioned at a front end portion and tension handle portion 432 extending past a back/rear end portion of the main housing 430. The main housing 430 may be shaped and sized to be manually engaged and manipulated.

The portion of the tension handle portion 432 extending past the back/rear end portion of the main housing 430 may form or comprise a handle knob portion 434 with a ring member or portion 433. The handle knob portion 434 may be shaped and sized to be manually engaged and manipulated. For example, as discussed further herein, the main housing 430 and the tension handle portion 432 may be manually longitudinally translated with respect to each, and the tension handle portion 432 may be rotated about its axis within the main housing 430 via the handle knob portion 434, to shorten the intermediate portion 356 of the tether 350 to bring the anchors 330, 310 towards each other (and in contact with bone and/or tissue), and to apply tension to the intermediate portion 356, to stabilize a joint.

As shown in FIGS. 40-49 and 51-60, the tension handle portion 432 comprises the rear handle knob portion 434 and a longitudinally elongated externally threaded shaft portion 436 extending longitudinally forward from the tension handle portion 432. The threaded shaft portion 436 extends through a rear aperture of the main housing 430 and longitudinally forward within the internal cavity of the main housing 430. The handle knob portion 434 is fixed with the threaded shaft portion 436 such that rotation of the handle knob portion 434 with respect to the main housing 430 (about a longitudinal axis thereof and/or of the threaded shaft portion 436) (e.g., manual rotation via the effectuates rotation of threaded shaft portion 436 with respect to (and at least partially within) the main housing 430.

The tension handle portion 432 may further comprise a longitudinally elongated release member or rod 437 with a rear engagement end 438 (that may be shaped or configured as a button or actuator) and a forward end, as shown in FIGS. 40-49 and 51-60. The release member or rod 437 is stiff, and extends through a cannulation, through hole/aperture or cavity of the handle knob portion 434 and the longitudinally elongated threaded shaft portion 436 with the rear engagement end 438 at or proximate to, and exposed in, the ring portion 433. The release member 437 is longitudinally movably retained within the cannulation of the handle knob portion 434 and the longitudinally elongated threaded shaft portion 436. In some embodiments, the release member or rod 437 may be resiliently biased, such as via a resilient member (e.g., a spring) (not shown), rearwardly such that the rear engagement end 438 is normally biased proud of a rear surface of the handle knob portion 434 such that it is exposed and manually accessible, such as in the ring portion 433, to translate the release member 437 longitudinally forward (toward the tip portion 429).

As shown in FIGS. 40-49 and 51-60, the tension handle portion 432 may also comprise a carrier portion at a longitudinal forward end of the tension handle portion 432 within the inner cavity of the main housing 430. The carrier portion and the inner cavity of the main housing 430 may be configured such that the carrier portion is able to longitudinally translate/move or slide within the tension handle portion 432, but is prevented from rotating (e.g., rotate about a longitudinal axis, such as a longitudinal axis of the main housing 430 and/or of the threaded shaft portion 436).

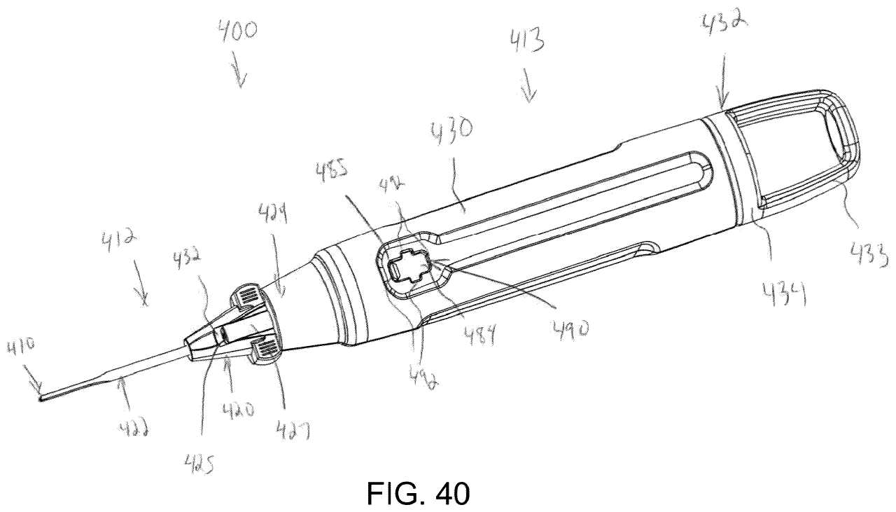

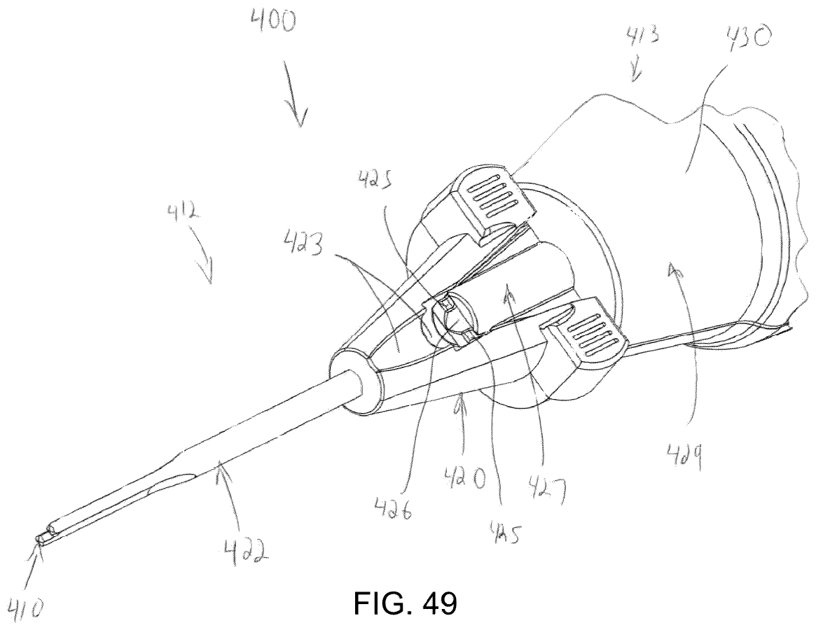

The carrier portion of the tension handle portion 432 may comprise a sleeve portion 440 that comprises at least one longitudinal aperture 441, and post portion 442 that comprises at least one post 443 that extends longitudinally rearwardly (away from the tip portion 429 of the instrument 400), as shown in FIG. 43. As shown in FIGS. 52-55 and 58-60, the sleeve portion 440 of the carrier portion is rotatably coupled and longitudinally fixed to a longitudinal forward end portion of the threaded shaft portion 436. In this way, longitudinal movement of the threaded shaft portion 436 effectuates longitudinal movement of the sleeve portion 440, but rotation of the threaded shaft portion 436 does not cause the sleeve portion 440 to rotate within the inner cavity of the main housing 430.

As also shown in FIGS. 52-55 and 58-60, the post portion 442 of the carrier portion is coupled to a longitudinal forward end portion of the release member 437. Longitudinal movement of the release member 437 thereby effectuates longitudinal movement of the post portion 442 within the inner cavity of the main housing 430. In this way, when the release member 437 is manually longitudinally translated forwardly (toward the tip portion 429) by a user via the engagement end 438 thereof, the post portion 442 is longitudinally translated forwardly with respect to the sleeve portion 440 (and the threaded shaft portion 436 and the main housing 430, for example).

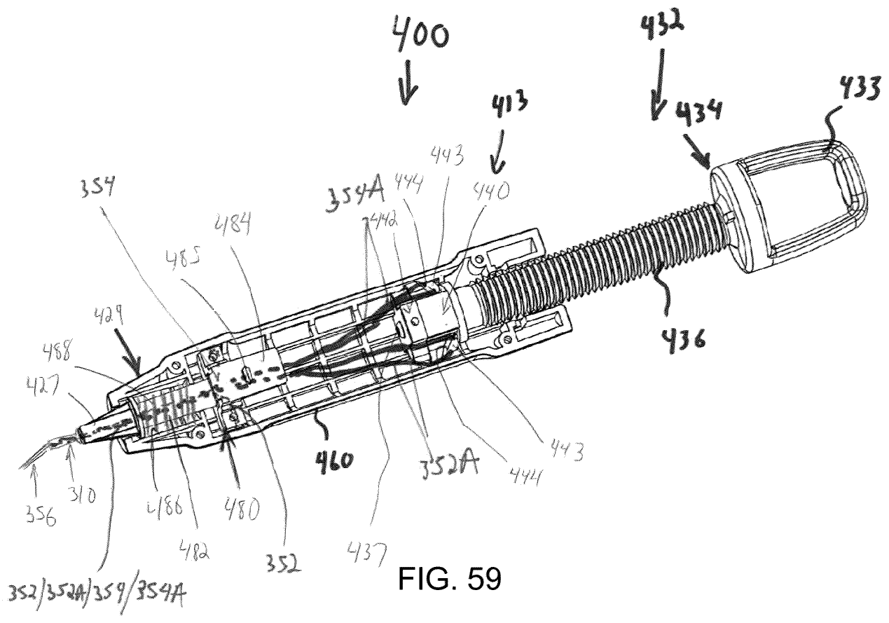

As shown in FIGS. 52-55 and 58-60, the sleeve portion 440 and the post portion 442 are movably assembled together/arranged together/coupled such that the at least one post 443 of the post portion 442 extends within the at least one aperture 441 of the post portion 442. As shown in FIGS. 52-55, 58 and 59, the post portion 442 is resiliently biased, such as via a resilient member (e.g., a spring) (not shown), longitudinally forwardly, into a first relative longitudinal arrangement. The sleeve portion 440 and the post portion 442 are configured such that in the first relative arrangement, the at least one post 443 of the post portion 442 extends through the at least one aperture 441 and past a rear engagement side or side surface/face 444 of the sleeve portion 440. However, the sleeve portion 440 and the post portion 442 can be rearranged into a second relative longitudinal arrangement via longitudinally forward translation of the release member 437 (e.g., via the engagement end 438), which forces the post portion 442 longitudinally forward with respect to the sleeve portion 440 such that the tip/end of the at least one post 443 is positioned below/beneath the engagement side 444 of the sleeve portion 440, as shown in FIG. 60.

It is noted that that the carrier portion, as a whole, is longitudinal fixed with the threaded shaft portion 436. That is, other than the relative movement between the post portion 442 and the sleeve portion 440 when biased into the second relative longitudinal arrangement from the first relative longitudinal arrangement, the post portion 442 and the sleeve portion 440 (and the release member 437) are longitudinally fixed or coupled at the forward end portion of the threaded shaft portion 436 such that longitudinal rearward translation of the threaded shaft portion 436 relative to the main housing portion 430 effectuates longitudinal rearward translation of the carrier portion (i.e., the post portion 442 and the sleeve portion 440) relative to the main housing portion 430 (within the main housing portion 430).

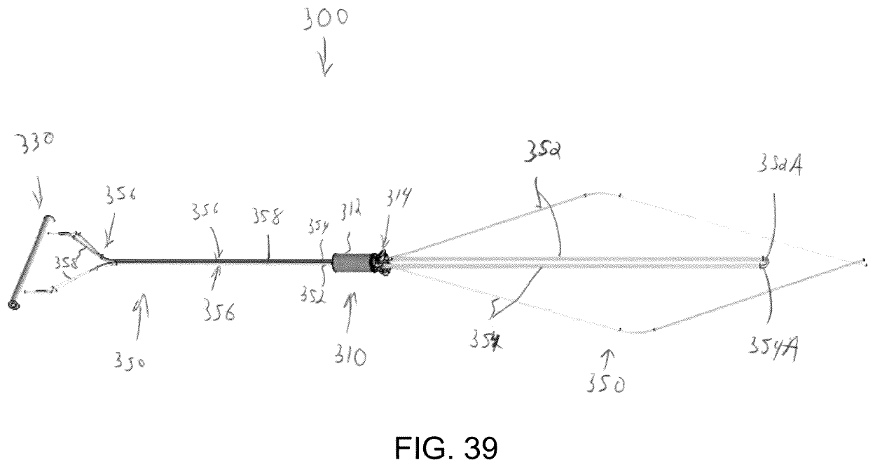

As shown in FIGS. 42-45, 52, 61 and 62, the threaded shaft portion 436 is threadably coupled with the main housing 430 such that rotation of the threaded shaft portion 436 about the longitudinal axis thereof and with respect to the main housing 430 causes the threaded shaft portion 436 to longitudinally translate in or out of the main housing 430 (depending upon the direction of rotation). In this way, the threaded shaft portion 436 can be rotated in a first rotational direction and with respect to the main housing 430 (such as via the rear handle knob portion 434) such that the threaded shaft portion 436 become less nested and the threaded shaft portion 436 longitudinally translates rearwardly with respect to the main housing 430 (and/or the main housing 430 longitudinally translates forwardly with respect to the threaded shaft portion 436).

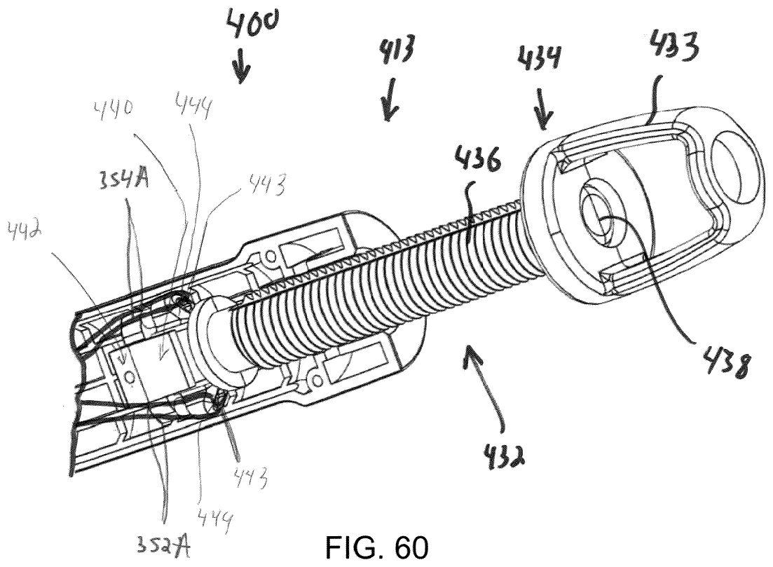

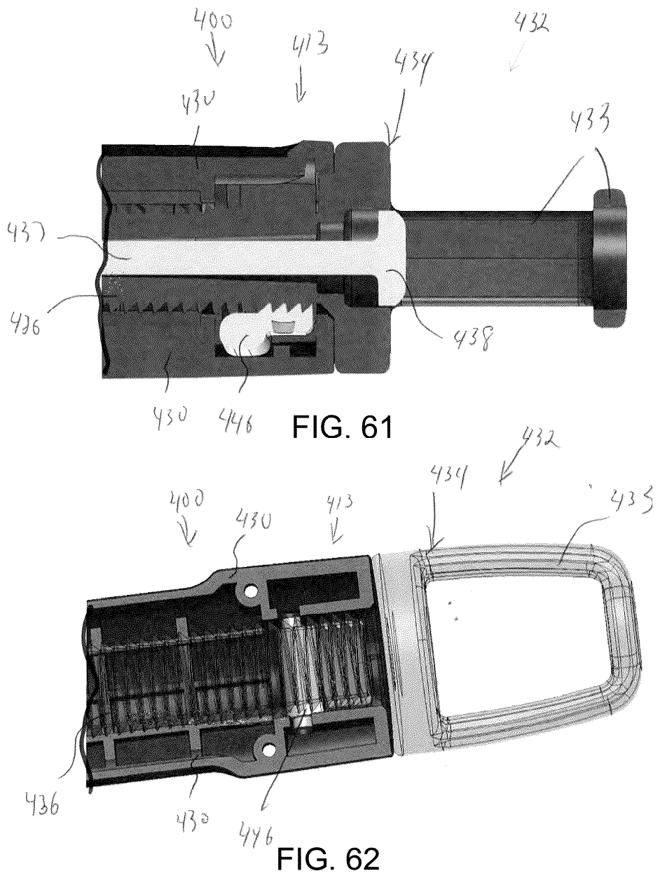

In some embodiments, the threaded shaft portion 436 is threadably coupled with the main housing 430 via internal threads or projections (e.g., teeth) of the main housing 430, such as within the inner cavity thereof. As shown in FIGS. 42-45, 52, 61 and 62, in some embodiments, the threaded shaft portion 436 is threadably coupled with the main housing 430 via at least one ratchet member 446 that is coupled to the main housing portion 430. The at least one ratchet member 446 may comprise threads or projections that mate with the external threads of the threaded shaft portion 436 to form a threaded connection. In some such embodiments, as shown in FIGS. 61 and 62, the at least one ratchet member 446 may be configured and resiliently biased (such as via a resilient member, such as a spring (not shown)) against the externally threaded shaft portion 436 such that the threaded shaft portion 436 is rotatably coupled and longitudinally rearwardly slidably coupled with the main housing portion 430. The at least one ratchet member 446 may be configured, such as via its configuration and/or range of movement, to provide for longitudinally rearward translation of the threaded shaft portion 436 with respect to the main housing portion 430, but prevent longitudinally forward translation of the threaded shaft portion 436 with respect to the main housing portion 430.

As shown in FIGS. 46, 47, 52-55, 58 and 59, in a preinstalled/installed, preconfigured/configured and/or loaded state/arrangement/configuration of the implant system, the first and second retention portions 352, 354 of the tether 350 extend through the aperture 426 of the tip member 427 and through at least a portion of the main housing portion 430, and are retained, fixed or otherwise coupled (e.g., releasably or fixedly) to the tension handle assembly 432. Longitudinal movement of the tension handle portion 432 relative to the main housing portion 430 in a first longitudinal direction extending away from the tip portion 429 (such as via the rear handle knob portion 434 and/or the threaded shaft portion 436)thereby pulls the first and second retention portions 352, 354 through the instrument 400 such that the head portion 314 of the dynamic bone anchor 310 is pulled against the tip portion 427/429 of the instrument 400, and once/if the dynamic bone anchor 310 is positioned against the tip portion 427/429, pulls the tether 350 through the dynamic bone anchor 310 and the soft bone anchor 330 to shorten the length of and/or apply tension to the intermediate portion 356 extending between the dynamic bone anchor 310 and the soft bone anchor 330.

In some embodiments, the first and second retention portions 352, 354 of the tether 350 are retained, fixed or otherwise coupled (e.g., releasably or fixedly) to the carrier portion of the tension handle assembly 432. Longitudinal movement of the carrier portion relative to the main housing portion 430 in a first longitudinal direction extending away from the tip portion 429 (such as via the rear handle knob portion 434 and/or the threaded shaft portion 436) thereby pulls the first and second retention portions 352, 354 through the instrument 400 such that the head portion 314 of the dynamic bone anchor 310 is pulled against the tip portion 427/429 of the instrument 400, and once/if the dynamic bone anchor 310 is positioned against the tip portion 427/429, pulls the tether 350 through the dynamic bone anchor 310 and the soft bone anchor 330 to shorten the length of and/or apply tension to the intermediate portion 356 extending between the dynamic bone anchor 310 and the soft bone anchor 330.

In some embodiments, the first and second retention portions 352, 354 of the tether 350 are retained, fixed or otherwise coupled (e.g., releasably or fixedly) to the post portion 442 of the carrier portion (such as to/with the at least one post 443 thereof). Longitudinal movement of the post portion 442 relative to the main housing portion 430 in a first longitudinal direction extending away from the tip portion 429 (such as via the rear handle knob portion 434 and/or the threaded shaft portion 436) thereby pulls the first and second retention portions 352, 354 through the instrument 400 such that the head portion 314 of the dynamic bone anchor 310 is pulled against the tip portion 427/429 of the instrument 400, and once/if the dynamic bone anchor 310 is positioned against the tip portion 427/429, pulls the tether 350 through the dynamic bone anchor 310 and the soft bone anchor 330 to shorten the length of and/or apply tension to the intermediate portion 356 extending between the dynamic bone anchor 310 and the soft bone anchor 330.

As shown in FIGS. 52-55, 58 and 59, in some embodiments, the first and second loop portions 352A, 354A of the first and second retention portions 352, 354 of the tether 350 may be retained, fixed or otherwise coupled, such as releasably retained, fixed or otherwise coupled, to the tension handle assembly 432. For example, in some embodiments, the first and second loop portions 352A, 354A may be retained, fixed or otherwise coupled, such as releasably retained, fixed or otherwise coupled, to the carrier portion of the tension handle assembly 432. In some such embodiments, the first and second loop portions 352A, 354A may be retained, fixed or otherwise coupled, such as releasably retained, fixed or otherwise coupled, to the post portion 442 of the carrier portion. For example, in some embodiments, the first and second loop portions 352A, 354A may be looped over the at least one post 443 of the post portion 442 of the carrier portion (i.e., the at least one post 443 extends within the interior of the respective loop) and extend over the engagement side 444 of the sleeve portion 440, as shown in FIGS. 52-55, 58 and 59.

As discussed above, in embodiments with the first and second loop portions 352A, 354A coupled to the at least one post 443 of the post portion 442 of the carrier portion, the first and second loop portions 352A, 354A may be released or decoupled from the post portion 442 via the by longitudinally forward translation of the release member 437 (e.g., via the engagement end 438), as shown in FIG. 60. A user may longitudinally translate the release member 437 forward toward the tip portion 427/429, such as via depressing the engagement end 438, to force the post portion 442 longitudinally forward with respect to the sleeve portion 440 such that the tip/end of the at least one post 443 is positioned below/beneath the engagement side 444 of the sleeve portion 440, as shown in FIG. 60. In such an arrangement, the first and second loop portions 352A, 354A are no longer looped over/about the at least one post 443, and are free to move off of the carrier portion. Once released from the at least one post 443, the first and second loop portions 352A, 354A may be translated as longitudinally forward by tensioning/pulling the free ends of the first and second retention portions 352, 354 longitudinally forward (such as by pulling/translating the instrument 400 longitudinally rearward) and reducing the size/length of the first and second loop portions 352A, 354A until they are reduced and/or tightened onto end portions of the first and second retention portions 352, 354 and the top side of the head portion 314 of the dynamic bone anchor 310.

In some embodiments, the first and second loop portions 352A, 354A may extend through the passageway 426 of the tip member 427 and through a portion of the internal cavity of the main housing portion 430 (which may include extending through one or more passageways, cannulations or apertures of a tension indicator assembly or portion) and to the tension handle assembly 432, such as to the carrier portion, such as to the post portion 442, such as about the at least one post 443 of the post portion 442 and over the engagement side 444 of the sleeve portion 440, as shown in FIGS. 52-55, 58 and 59.

In some embodiments, the end portions of the first and second retention portions 352, 354 may be retained, fixed or otherwise coupled (releasably or fixedly) to a portion of the instrument 400, as shown in FIGS. 52-55, 58 and 59. In some such embodiments, the portions of the first and second retention portions 352, 354 may not be coupled to the tension handle portion 432, as shown in FIGS. 52-55, 58 and 59. For example, in some embodiments, the end portions of the first and second retention portions 352, 354 may be retained, fixed or otherwise coupled directly or indirectly to the main housing portion 430, such as within the inner cavity of the main housing portion 430, as shown in FIGS. 52-55, 58 and 59. In some such embodiments, the end portions of the first and second retention portions 352, 354 may be coupled (releasably or fixedly) to a portion of the tension indicator assembly or portion, as shown in FIGS. 52-55, 58 and 59. In some other embodiments, the end portions of the first and second retention portions 352, 354 may be coupled (releasably or fixedly) to the tension handle assembly 432, such as to the carrier portion, such as to the post portion 442 and/or the sleeve portion 440. In some embodiments, the first and second end portions of the first and second retention portions 352, 354 may extend through the passageway 426 of the tip member 427 and through a portion of the internal cavity of the main housing portion 430 (which may include extending through one or more passageways, cannulations or apertures of the tension indicator assembly or portion), as shown in FIGS. 52-55, 58 and 59.

The instrument 400 is thereby configured such that longitudinal translation of the tension handle portion 432 (e.g., at least the carrier portion and/or post portion 442 thereof) rearwardly relative to the main housing portion 430 tensions the first and second retention portions 352, 354 (in a longitudinal rearward direction). Rearward longitudinal translation of the tension handle portion 432 relative to the main housing portion 430 pulls the tether 350 through the instrument 400 (e.g., the tip member 427 and main housing portion 430) to pull/draw and seat the dynamic bone anchor 310 (such as the head portion 314 thereof) against the instrument 400 (such as against the forward tip of the tip member 427 (if not already seated/retained thereon/thereagainst)), as shown in FIGS. 52-55, 58 and 59. Further, if the dynamic bone anchor 310 is seated/retained on/against a tip portion 427/429 of the instrument 400 (e.g., the forward tip of the tip member 427), rearward longitudinal translation of the tension handle portion 432 relative to the main housing portion 432 draws/pulls/translates the tether 350 through the dynamic bone anchor 310 and the and the soft bone anchor 330 to shorten the length of the intermediate loop portion 356, which thereby draws the dynamic bone anchor 310 and the soft bone anchor 330 towards each other (which may seat the dynamic bone anchor 310 and/or the soft bone anchor 330 against bones and/or bone plates or other hardware attached to bones, and/or move the bones) and/or applies tension to the intermediate loop portion 356 extending between the dynamic bone anchor 310 and the soft bone anchor 330 (and thereby compresses or constrains the joint between the bones)). Longitudinal movement of the tension handle portion 432 relative to the main housing portion 430 in a first longitudinal direction extending away from the tip portion 429 (such as via the rear handle knob portion 434 and/or the threaded shaft portion 436) may thereby pull the first and second loop portions 352A, 354A through the instrument 400 such that the head portion 314 of the dynamic bone anchor 310 is pulled against the tip portion 427/429 of the instrument 400, and once/if the dynamic bone anchor 310 is positioned against the tip portion 427/429, enlarge the first and second loop portions 352A, 354A by pulling the of the tether 350 through the dynamic bone anchor 310 and the soft bone anchor 330 to shorten the length of and/or apply tension to the intermediate portion 356 extending between the dynamic bone anchor 310 and the soft bone anchor 330, as shown in FIGS. 52-55, 58 and 59.

As discussed herein above, longitudinal translation of the tension handle portion 432 (e.g., at least the carrier portion and/or post portion 442 thereof) rearwardly relative to the main housing portion 430 can be done manually via manually longitudinally sliding the threaded shaft portion 436 (such as by manually longitudinally pulling on the rear handle knob portion 434) and/or manually rotating the threaded shaft portion 436 (such as by rotating the rear handle knob portion 434) in a first rotational direction to threadably longitudinally move/translate the tension handle portion 432 rearwardly relative to the main housing portion 430.

It is noted that any tension applied to the intermediate loop portion 356 of the tether 350 between the between the dynamic bone anchor 310 and the soft bone anchor 330 is retained after the first and second retention portions 352, 354 are decoupled or released from the instrument 400 (e.g., from the post portion 442) and prior to the first and second loop portions 352A, 354A being reduced and/or tightened onto end portions of the first and second retention portions 352, 354 and the top side of the head portion 314 due to the nature/configuration of the implant system 300. For example, the tether 350 experiences a substantial amount of friction against itself and the soft bone anchor 330 as it extends therethrough (several times), and against itself and the components of the dynamic bone anchor 310 (e.g., the anchor body 312, the deformable bumper 318, the tether post 320 and the head portion 314) as it extends through the cannulation and passageways thereof (several times). At least such friction is sufficient to prevent the tether 350 from slipping through the soft bone anchor 330 and/or the dynamic bone anchor 310, and thereby enlarging the intermediate loop portion 356 between the soft bone anchor 330 and the dynamic bone anchor 310 and, thereby, losing/reducing any tension thereof.

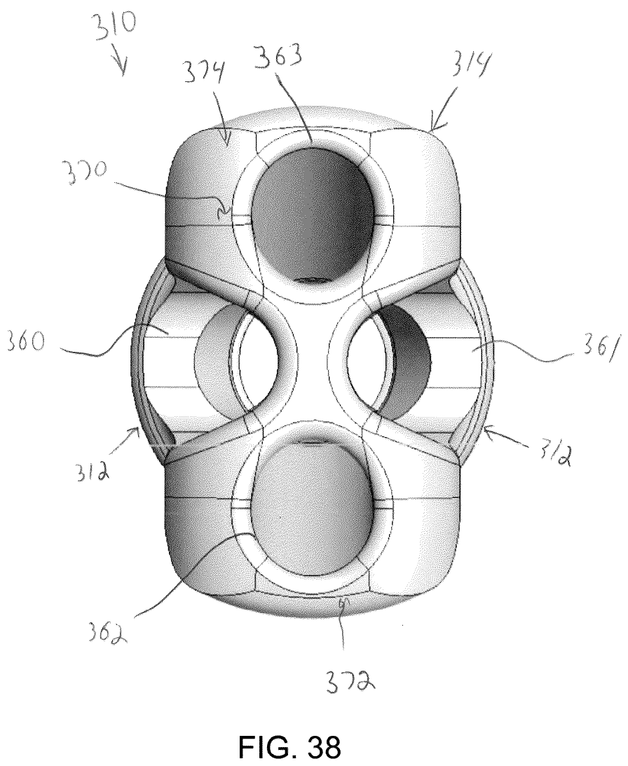

As shown in FIGS. 40-43, 45-48 and 52-59, the instrument 400 may include a tension indicator feature, as discussed above. The tension indicator feature provides a user a visual indication of whether or not a tension above a threshold was applied to the tether 350 (the intermediate loop portion 356) between the soft bone anchor 330 and the dynamic bone anchor 310 via the instrument 400 (and a degree as to how far a tension is above the threshold). The tension indicator feature may comprise a tension indicator assembly that mates with the main housing portion 430 and the dynamic bone anchor 310, and a reference portion of the main housing portion 430, as shown in FIGS. 41, 42, 43, 45, 52-55, 58 and 59. The tension indicator feature may be configured to utilize the tension of the first and second retention portions 352, 354 that is applied to the dynamic bone anchor 310, to effectuate a tension of the intermediate loop portion 356, to provide the visual indication.

As shown in FIGS. 41, 42, 43, 45, 52-55, 58 and 59, the tension indicator assembly may comprise the tip member 427, a resilient/elastic member (e.g., a spring or bumper) 488 and an indicator body 480, as shown in FIGS. 41, 42, 43, 45, 52-55, 58 and 59. The indicator body 480 may include an indicator portion 484 with a visual indication 485, and a body member or portion 482. As also shown in FIGS. 41, 42, 43, 45, 52-55, 58 and 59, a front portion of the main housing portion 430 may include an inner indicator cavity or recess 486 that extends laterally and longitudinally between forward and rear walls or partitions.

As also shown in FIGS. 41, 42, 43, 45, 52-55, 58 and 59, the tip member 427 extends into indicator cavity 486, and the resilient member 488 is positioned longitudinally between the rear wall of the indicator cavity 486 and the tip member 427. The resilient member 488 biases or positions the tip member 427 longitudinally forward against the forward wall of the indicator cavity 486. The tip member 427 is thereby longitudinally movably coupled within the main housing portion 430 (within the indicator cavity 486), and the resilient member 488 resists movement of the tip member 427 relative to the main housing portion toward a rear portion of the main housing portion 430 when the dynamic bone anchor 310 is against the tip/end of the tip member 427 and the first and second retention portions 352, 354 are tensioned by the tension handle portion 432.

The body member or portion 482 of the indicator body 480 may extend longitudinally through at least a portion of the resilient member 488, and may abut or be coupled to the tip member 427. Rearward longitudinal movement of the tip member 427 thereby effectuates rearward longitudinal movement of the indicator body 480. As shown in FIGS. 41, 42, 43, 45, 52-55, 58 and 59, in some embodiments the body member or portion 482 of the indicator body 480 may comprise a tube or like member that defines an inner passageway and that extends longitudinally through at least a portion of the resilient member 488. The first and second retention portions 352, 354 (e.g., the end portions of the first and second retention portions 352, 354 and/or the intermediate loop portions 352A, 354A of the first and second retention portions 352, 354) may extend longitudinally from the head portion 314 of the dynamic bone anchor 310 and into the inner cavity of the tension handle portion 432/instrument 400 by extending longitudinally through the passageway 426 in the tip member 427 and the inner passageway the body member or portion 482 of the indicator body 480, as shown in FIGS. 41, 42, 43, 45, 52-55, 58 and 59.

As shown in FIGS. 52-55, 58 and 59, the end portions of the first and second retention portions 352, 354 of the tether 350 may be coupled (e.g., fixedly coupled) to the tension indicator assembly, such as to the indicator body 480. Accordingly, when the first and second retention portions 352, 354 of the tether 350 are under tension, and thereby applying such tension to the intermediate loop portion 356 between the soft bone anchor 330 and the dynamic bone anchor 310, the tension of the end portions of the first and second retention portions 352, 354 pulls/tensions the indicator body 480 longitudinal forward, such as against the forward wall of the indicator cavity 486.

As shown in FIGS. 40-43, 45-48 and 52-59, the indicator portion 484 with the visual indication 485 of the indicator body 480 may be positioned within the inner cavity of the tension handle portion 432/instrument 400 just below/beneath the outer wall of the tension handle portion 432. As also shown in FIGS. 40-43, 45-48 and 52-59, the tension handle portion 432 includes an indicator window 490 (e.g., an opening aligned with the indicator portion 484) with at least one visual reference portion or indication 492 such that the indicator portion 484 with the visual indication 485 of the indicator body 480 is exposed therethrough.

The tension indicator feature is configured such that when the head portion 314 of the dynamic bone anchor 310 is retained/adjacent/abutting the tip/end of the tip member 427, and tension is applied to intermediate loop portion 356 between the soft bone anchor 330 and the dynamic bone anchor 310 via tension of the first and second retention portions 352, 354 (e.g., the end portions of the first and second retention portions 352, 354 and/or the intermediate loop portions 352A, 354A of the first and second retention portions 352, 354), the tip portion 427 is being pulled/tensioned longitudinally rearwardly against the resilient member 488. If such tension force is below the strength/elasticity/spring constant of the resilient member 488, the tip portion 427, and thus the indicator body 480 (with the indicator portion 484/visual indication 485), will not translate longitudinally rearwardly, and the visual indication 485 will be positioned longitudinally forward within the indicator window 490 and in relation to the at least one visual reference portion or indication 492, as illustrated in FIGS. 52-56.

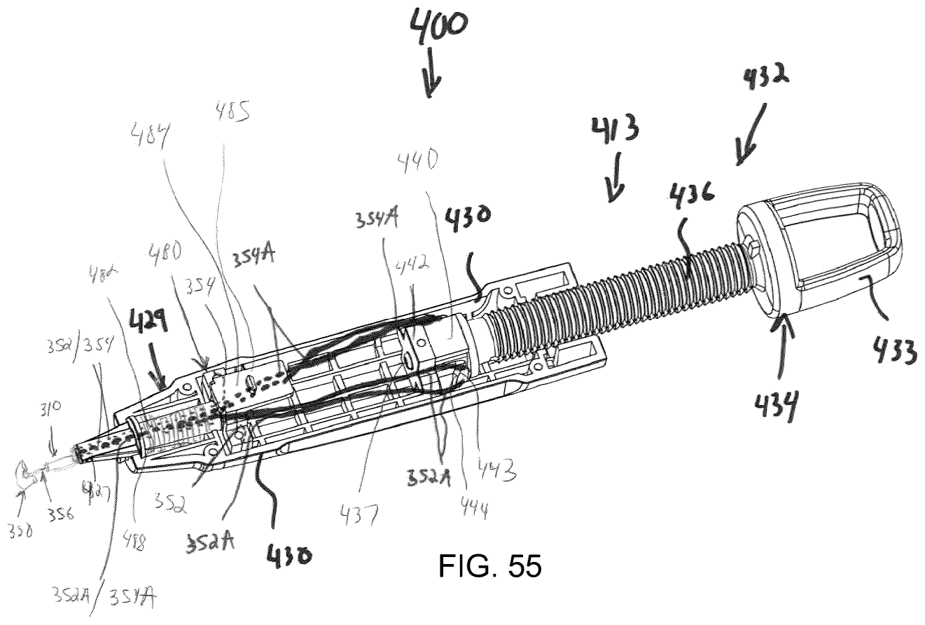

However, if the longitudinally rearward tension force applied to the tip portion 427 via the dynamic bone anchor 310 by the first and second retention portions 352, 354 (e.g., the end portions of the first and second retention portions 352, 354 and/or the intermediate loop portions 352A, 354A of the first and second retention portions 352, 354) is greater than the strength/elasticity/spring constant of the resilient member 488, the tip portion 427, and thus the indicator body 480 (with the indicator portion 484/visual indication 485), will translate longitudinally rearwardly, and the visual indication 485 will move longitudinally forwardly within the indicator window 490 and in relation to the at least one visual reference portion or indication 492, as illustrated in FIGS. 57-59. The strength/elasticity/spring constant of the resilient member 488, and/or the indicator window 490 and the at least one visual reference portion or indication 492, may thereby be configured or selected to correlate to a particular tension force threshold (or force range threshold). The particular strength/elasticity/spring constant of the resilient member 488 (and/or location of the indicator window 490 and the at least one visual reference portion or indication 492) thereby acts as a tension threshold above which must be applied to intermediate loop portion 356 between the soft bone anchor 330 and the dynamic bone anchor 310 via the tension of the first and second retention portions 352, 354 (e.g., the end portions of the first and second retention portions 352, 354 and/or the intermediate loop portions 352A, 354A of the first and second retention portions 352, 354) for the visual indication 485 to move in relation to the indicator window 490 and to the at least one visual reference portion or indication 492, thus providing an indication to the user of the tension applied to the intermediate loop portion 356 (and thereby across a joint, for example).

To dynamically stabilize a distal tibiofibular syndesmosis joint, the instrument 400 may be used to implant the implant system 100 or 300 across the joint. For example, with reference to the implant system 300 for illustration purposes only, the implant system 300 may be loaded/retained/configured on the instrument 400. The soft bone anchor 330 may be retained on the forked free end 410 of the first portion 412 of the instrument 400, and the head portion 314 of the dynamic bone anchor 410 may be retained on the recess 425 of the tip member 427 and within the recess/cavity formed by the inner lateral side portion 423 of the base portion 420 of the first portion 412. The first and second retention portions 352, 354 (e.g., the end portions of the first and second retention portions 352, 354 and/or the intermediate loop portions 352A, 354A of the first and second retention portions 352, 354) extend into the instrument 400 via the passageway 426 of the tip member 427 and the passageway of the indicator body 480.

The end portions of the first and second retention portions 352, 354 are fixed to the instrument 400, such as to the indicator body 480. The first and second intermediate loop portions 352A, 354A extend within the main housing portion 430 to the carrier portion, and are releasably coupled thereto. The first intermediate loop portion 352A is looped over a first post 443 of the post portion 442, and extends on/over the engagement surface 444 of the sleeve portion 440, and the second intermediate loop portion 354A is looped over a second post 443 of the post portion 442, and extends on/over the engagement surface 444 of the sleeve portion 440. The first and second retention portions 352, 354, and potentially the intermediate loop portion 356, may be under some amount of tension so as to relatively securely retain the soft bone anchor 330 on the forked free end 410 and the head portion 314 of the dynamic bone anchor 410 on the recess 425 of the tip member 427.

With the implant system 300 mounted and retained on the instrument 400, the instrument 400 may be manually manipulated/maneuvered such that the soft bone anchor 330 is introduced into, and passed through, a hole (e.g., drilled hole) in a fibula, and ultimately positioned within a hole in an adjacent tibia or past an outer surface of the tibia.

After the soft bone anchor 330 is inserted into a desired position in/on the tibia, the instrument may be manually pulled backward/rearward in a direction opposing the direction of insertion, such as my manually grasping the tip portion 429 of the instrument 4000 and pulling in the backward/rearward direction. Such backward/rearward force/movement deforms the soft bone anchor 330 into an enlarged size such that it is retained securely within/on the tibia. The backward/rearward force/movement may also remove/decouple the soft bone anchor 330 from the forked free end 410.

Once the soft bone anchor 330 is securely implanted, the first portion 412 of the instrument 400 may be removed from the second portion 113. For example, the second portion 113 may be manually laterally translated/slid (towards the exposed internal tether 350) with respect to the first portion to release the second portion 113 from the first portion 112 and to expose the dynamic bone anchor 310. After it is disconnected, the first portion 112 can be removed from the patient (e.g., from the fibula) by pulling directly back from the limb.

After the first portion 112 is disconnected from the second portion 113, and the first portion 112 is removed from the patient, the instrument 400 can be used to implant the dynamic bone anchor 310 into the fibula. For example, the while maintaining tension on the dynamic bone anchor 310 from the intermediate loop portion 356 by pulling/holding the loop/ring 433 of the handle knob portion 434 of the tension handle portion 432 backward/rearward, the user can manually slidably press/translate the main housing portion 430 longitudinally forwards relative to the tension handle portion 432 towards the fibula to advance the dynamic bone anchor 310 towards the fibula. As the dynamic bone anchor 310 approaches the fibula (which may include a bone plate coupled to the fibula), the anchor body 312 of dynamic bone anchor 310 may be inserted into the hole in the fibula and/or bone plate, and the dynamic bone anchor 310 further longitudinally slidably advanced until the head portion 314 is seated against the fibula and/or bone plate.

With the dynamic bone anchor 310 seated against the fibula and/or bone plate, the tension handle portion 432 may be manually rotated relative to the relative to the tension handle portion 432 (such as via the loop/ring 433 of the handle knob portion 434) a first rotational direction (e.g., clockwise or counterclockwise) for fine adjustment of the size/length of the intermediate loop portion 356 to adjust the distance between the soft bone anchor 330 and the dynamic bone anchor 310, and thereby adjust the position of the dynamic bone anchor 310 relative to the fibula/bone plate and/or apply a particular amount of tension to the intermediate loop portion 356 (and thereby across the joint between the tibia and fibula). For example, the tension handle portion 432 can be longitudinally adjusted (slidably and/or rotationally) relative to the main housing portion 430 such that the tension applied to the intermediate loop portion 356 (and thereby across the joint between the tibia and fibula) via the first and second retention portions 352, 354 reaches the tension threshold of the tension indicator feature such that the tension indicator feature indicates that a proper or appropriate level of tension is present.

After the dynamic bone anchor 310 is seated and the tension of the intermediate loop portion 356 is set, the user can depress the rear engagement end 438 of the release member 437 to actuate the release member 437 and release the first and second intermediate loop portions 352A, 354A from the posts 443 of the post portion 442 of the carrier portion. With the first and second intermediate loop portions 352A, 354A released, the user can pull the instrument 400 away from the limb to elongate the end portions of the first and second retention portions 352, 354 and reduce the first and second intermediate loop portions 352A, 354A onto the end portions of the first and second retention portions 352, 354 and the head portion 314 of the dynamic bone anchor 310. Once the first and second intermediate loop portions 352A, 354A are reduced down onto the end portions of the first and second retention portions 352, 354 and the head portion 314 of the dynamic bone anchor 310, the first and second retention portions 352, 354 may be manually pulled/tensioned in directions opposing each other (such as approximately in line with the axis of the fibula) to securely couple the first and second retention portions 352, 354 to the head portion 314 of the dynamic bone anchor 310. If desired, the first and second retention portions 352, 354 may, or may not, then be knotted (e.g., via one or more surgical knots). Excess length the first and second retention portions 352, 354 may finally be trimmed from the construct.

As may be recognized by those of ordinary skill in the art based on the teachings herein, numerous changes and modifications may be made to the above-described and other embodiments of the present disclosure without departing from the scope of the disclosure. In addition, the implants and systems may include more or fewer components or features than the embodiments as described and illustrated herein. Accordingly, this detailed description of the currently-preferred embodiments is to be taken in an illustrative, as opposed to limiting of the disclosure.

Similarly, positions or directions may be used herein with reference to anatomical structures or surfaces. Further, the implants, systems, devices, instrumentation and methods, and the aspects, components, features and the like thereof, may be disclosed herein are described with respect to one side of the body for brevity purposes. However, as the human body is relatively symmetrical or mirrored about a line of symmetry (midline), it is hereby expressly contemplated that the implants, systems, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems, instrumentation and methods, and the aspects, components, features and the like thereof, described herein with respect to a right syndesmosis joint (right ankle/leg) may be mirrored or otherwise reconfigured, if necessary or desirable, so that they likewise function with a left syndesmosis joint (left ankle/leg). Further, the implants, systems, devices, instrumentation and methods, and the aspects, components, features and the like thereof, disclosed herein are described with respect to a distal tibiofibular syndesmosis, but it should be understood that the implants, systems, devices, instrumentation and methods may be used with other bones of the body having similar structures.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An implant system for dynamic stabilization of a syndesmosis joint, comprising:
a soft bone anchor;
a dynamic bone anchor; and
a tether coupled to and extending between the soft bone anchor and the dynamic bone anchor,
wherein first and second retention portions of the tether are knotlessly coupled to the dynamic bone anchor, and an intermediate portion of the tether extending between the first and second retention portions passes through the soft bone anchor, and
wherein a medial portion of the intermediate portion extends into the dynamic bone anchor and is resiliently coupled to the dynamic bone anchor.

2. The system according to claim 1, wherein the soft anchor is a suture anchor configured to deform into a dense enlarged shape upon tensioning of the intermediate portion of the tether, and wherein the tether is a suture.

3. The system of according to claim 1, wherein the dynamic bone anchor comprises a head portion at one end and a body portion at an opposing end, and wherein the head portion and the body portion are cannulated, and wherein the first and second retention portions pass through the cannulation of the head portion and the body portion, and passes out apertures of the head portion.

4. The system according to claim 3, wherein the dynamic bone anchor further comprises a resilient bumper and a movable tether post within the cannulation of the body portion, and wherein the resilient bumper and the tether post are cannulated, and wherein the first and second retention portions pass through the cannulation of the resilient bumper and the tether post.

5. The system according to claim 4, wherein the resilient bumper and the tether post are cannulated, and wherein the first and second retention portions pass through the cannulation of the resilient bumper and the tether post, wherein the resilient bumper is retained between an end of the body portion and the tether post, and wherein the medial portion of the intermediate portion extends about a portion of the tether post and through the cannulation of the resilient bumper.

6. The system according to claim 5, wherein the first retention portion extends through cannulation of the head portion, out a first aperture of the head portion, over a portion of the outer top side, down into a second aperture of the head portion into the cannulation thereof, and back up through a fourth aperture of the head portion.

7. The system according to claim 6, wherein the second retention portion extends through cannulation of the head portion, out the second aperture of the head portion, over a portion of the outer top side, down into the first aperture of the head portion into the cannulation thereof, and back up through a third aperture of the head portion.

8. The system according to claim 7, wherein the portions of the first and second retention portions that extend over the portions of the outer top side form first and second retention loops, respectively, positioned past the outer top side of the head portion, and wherein the portions of the first and second retention portions that extend from the fourth and third apertures, respectively, pass under at least one of the first and second retention loops.

9. The system according to claim 6, wherein the first and second apertures are open at respective lateral sides of the head portion, and the third and fourth apertures are enclosed at the top side of the head portion by the top side of the head portion.

10. The system according to claim 3, wherein an enlarged top portion of the head portion comprises an oblong cross-sectional shape, and wherein the enlarged top portion of the head portion comprises first and second lateral side portions that extend laterally past a base portion of the head portion and comprise a tapered underside, and third and fourth lateral side portions that extend between the first and second sides that do not extend laterally past the base portion.

11. The system according to claim 1, further comprising an insertion and tensioning instrument, the soft bone anchor being removably retained on a free end of a first portion of the instrument, and the dynamic bone anchor being removably retained on at least one of the first portion and a second portion of the instrument, and wherein the first portion is removably coupled to the second portion.

12. The system according to claim 11, wherein the first portion comprises a base portion that is removably coupled to a tip portion of the second portion, and a longitudinally elongated inserter portion extending longitudinally from the base portion to the free end, and wherein the free end of the inserter portion is a forked free end that comprises a pair of tines and a base retention portion extending therebetween, and a pair of grooves extending longitudinally proximally from the base retention portion of the forked free end.

13. The system according to claim 11, wherein a head portion of the dynamic bone anchor is retained on a recess of a tip portion of the second portion, wherein a base portion of the first portion extends partially over the recess on the tip portion of the second portion when the first portion is removably coupled to the second portion, and wherein at least a portion of at least the head portion is positioned between the recess and the base portion when the first portion is removably coupled to the second portion to removably retain the dynamic bone anchor on the instrument.

14. The system according to claim 11, wherein the second portion of the instrument comprises a main housing portion and a tension handle portion manually movably coupled with the main.

15. The system according to claim 14, wherein the tension handle portion is longitudinally slidably coupled with the main housing portion, and wherein longitudinal translation of the tension handle portion in a first direction extending away from a tip portion of the second portion of the instrument pulls the tether through the main housing portion to pull the dynamic bone anchor against the tip portion.

16. The system according to claim 14, wherein the tension handle portion is longitudinally slidably coupled with the main housing portion, and wherein longitudinal translation of the tension handle portion in a first longitudinal direction extending away from a tip portion of the second portion of the instrument pulls the tether through the dynamic bone anchor and the soft bone anchor when the dynamic bone anchor is pulled against the tip portion to shorten the length of and/or tension the intermediate portion extending between the dynamic bone anchor and the soft bone anchor.

17. The system according to claim 15, wherein the second portion of the instrument further comprises at least one ratchet member coupled to the main housing portion comprising at least one tooth, the at least one ratchet member being resiliently biased against an externally threaded shaft portion of the tension handle portion such that the tension handle portion is rotatably coupled and longitudinally slidably coupled with the main housing portion, and wherein the at least one ratchet member prevents the tension handle portion from longitudinally slidably translating with respect to the main housing portion along a second longitudinal direction that opposes the first longitudinal direction.

18. The system according to claim 14, wherein the first retention portion of the tether forms a first loop portion extending from a head portion of the dynamic bone anchor past an outer top side of the head portion, and a first end portion extending from the head portion past the outer top side, and wherein the second retention portion of the tether forms a second loop portion extending from the head portion past the outer top side, and a second end portion extending from the head portion past the outer top side.

19. The system according to claim 18, wherein the first end portion of the first retention portion of the tether extends over the outer top side and through the first loop portion and the second loop portion, and wherein the second end portion of the first retention portion of the tether extends over the outer top side and through the first loop portion and the second loop portion to knotlessly couple the first and second retention portions to the dynamic bone anchor.

20. The system according to claim 18, wherein the first retention portion of the tether extends through a first aperture of the head portion from an internal passageway of the head portion, over a portion of the outer top side, and back into the internal passageway via a second aperture of the head portion to form the first loop portion, and wherein the first retention portion of the tether extends through the internal passageway from the second aperture, and through a third aperture of head portion past the outer top side to form the first end portion, and wherein the second retention portion of the tether extends through the second aperture of the head portion from the internal passageway, over a portion of the outer top side, and back into the internal passageway via the first aperture of the head portion to form the second loop portion, and wherein the second retention portion of the tether extends through the internal passageway from the first aperture, and through a fourth aperture of head portion past the outer top side to form the second end portion.

21. The system according to claim 18, wherein the first and second loop portions extend through a tip portion of the second portion and through a portion of the main housing portion to a carrier portion of the tension handle portion, and wherein the first and second loop portions are releasably coupled with the carrier portion.

22. The system according to claim 21, wherein movement of the tension handle portion relative to the main housing portion in a first direction extending away from the tip portion effectuates movement of the carrier portion in the first direction extending away from the tip portion to enlarge the first and second loop portions and pull the tether through the dynamic bone anchor and the soft bone anchor when the dynamic bone anchor is pulled against the tip portion to shorten the length of and/or tension the intermediate portion extending between the dynamic bone anchor and the soft bone anchor.

23. The system according to, claim 21 wherein the carrier portion comprises a sleeve portion within the main housing portion that comprises at least one aperture, and post portion within the main housing portion that comprises at least one post that extends in a first direction extending away from the tip portion, and wherein the sleeve portion and the post portion are biased into a first relative arrangement such that the at least one post extends through the at least one aperture and past an engagement side of a sleeve portion.

24. The system according to claim 23, wherein the first and second loop portions extend around the at least one post and over the engagement side of the sleeve portion, and wherein the carrier portion further comprises a release member that is exposed at a back end of the tension handle portion and extends to the post portion, and wherein the tension handle portion is configured such that translation of the release member in a second direction that opposes the first direction translates the post portion relative to the sleeve portion in the second direction such that the at least one post is positioned beneath the engagement side of the sleeve portion to release the first and second loop portions from the at least one post.

25. The system according to claim 14, wherein the first and second retention portions are coupled to the tension handle portion, and wherein the instrument is configured such that translation of the tension handle portion relative to the main housing portion tensions the first and second retention portions, wherein the instrument is configurated such that the tensioning of the first and second retention portions via the tension handle portion pulls a head portion of the dynamic bone anchor against a tip portion of the second portion, and pulls the tether through the dynamic bone anchor and the soft bone anchor to shorten the length of and tension the intermediate portion extending between the dynamic bone anchor and the soft bone anchor.

26. The system according to claim 25, wherein the tip portion of the second portion comprises a tip member longitudinally movably coupled with a front portion of the main housing portion, and wherein the second portion further comprises a resilient member configured to resiliently resist movement of the tip portion relative to the main housing portion toward a rear portion of the main housing portion when the dynamic bone anchor is against the tip portion and the first and second retention portions are tensioned by the tension handle portion.

27. The system according to claim 25, wherein the second portion further comprises a tension indicator longitudinally translatably coupled with the main housing portion comprising an indicator portion with a visual indication that is visually exposed and adjacent to a reference portion of the main housing portion, and wherein the tension of the first and second retention portions above a threshold longitudinally translates the visual indication relative to the reference portion to provide a visual indication of the tension of the first and second retention portions, and thereby a visual indication of the tension of the intermediate portion extending between the dynamic bone anchor and the soft bone anchor

28. The system according to claim 14, wherein first and second loop portions of the first and second retention portions, respectively, are coupled to the tension handle portion, and wherein the instrument is configured such that translation of the tension handle portion relative to the main housing portion tensions the first and second retention portions, and wherein first and second end portions of the first and second retention portions, respectively, are fixed to the instrument.

29. The system according to claim 28, wherein the first and second end portions are fixed to the instrument within the main housing portion, and are not fixed to the tension handle portion.

30. The system according to claim 14, wherein a back end portion of the tension handle portion comprises a handle knob portion with a ring portion.
